(19) United States
(12) Patent Application Publication
(10) Pub. No.: US 2022/0237906 A1
(43) Pub. Date: Jul. 28, 2022

(54) DETERMINATION DEVICE, DETERMINATION METHOD, AND PROGRAM

(71) Applicant: LIXIL Corporation, Tokyo (JP)

(72) Inventors: Emi UEDA, Tokyo (JP); Nobuhiro TAKI, Tokyo (JP); Kenta TANAKA, Tokyo (JP); Yasuhiro SHIRAI, Tokyo (JP); Hiroshige AOYAMA, Tokyo (JP); Toshiaki SHIMAZU, Tokyo (JP)

(73) Assignee: LIXIL Corporation, Tokyo (JP)

(21) Appl. No.: 17/611,897

(22) PCT Filed: May 15, 2020
(86) PCT No.: PCT/JP2020/019422

§ 371 Date: Nov. 16, 2021

(30) Foreign Application Priority Data
May 17, 2019 (JP) 2019-093674
Nov. 28, 2019 (JP) 2019-215658

(51) Int. Cl. G06V 10/82; G06V 10/774; G06V 20/50; E03D 9/00

(52) U.S. Cl. CPC G06V 10/82; E03D 9/00; G06V 20/50; G06V 10/7747

(57) ABSTRACT

A determination device includes an image information acquirer configured to acquire image information of a subject image obtained by photographing an internal space of a toilet bowl in excretion; an estimator configured to perform estimation regarding a determination matter relating to excretion by inputting the image information to a learned model, the learned model having learned a correspondence relationship between an image for learning and a determination result of the determination matter relating to excretion, the learned model learned by machine learning using a neural network, the image for learning representing an internal space of a toilet bowl in excretion; and a determiner configured to perform determination regarding the determination matter of the subject image based on an estimation result obtained by the estimator.

### CROSS REFERENCE TO RELATED APPLICATIONS

This application is a national stage application under 35 USC 371 of International Application No. PCT/JP2020/019422, filed May 15, 2020, which claims the priority of Japanese Application No. 2019-093674, filed May 17, 2019 and Japanese Application No. 2019-215658, filed Nov. 28, 2019, the entire contents of each of which are incorporated herein by reference.

### FIELD OF THE DISCLOSURE

This disclosure relates to a determination device, a determination method, and a program.

### BACKGROUND OF THE DISCLOSURE

An attempt to grasp the situation of excretion in a biological body is known. For example, the technology of photographing excrement by a camera and analyzing the photographed image is disclosed (for example, refer to Patent Document 1).

Machine learning is generally used to, for example, analyze an expression of a person. The technique using machine learning involves, for example, executing machine learning by using learning data that associates a feature in an expression of a person with a feeling corresponding to the expression, to thereby create a learned model. A feature in an expression of a person is input to the learned model, which enables the learned model to estimate a feeling indicated by the expression and analyze the expression.
PATENT DOCUMENT 1 Japanese Patent Application Laid-Open No. 2007-252805

### SUMMARY OF THE DISCLOSURE

The technology as described in Document 1 does not perform analysis with sufficient accuracy. In other words, the technology as described in Document 1 performs analysis by using a table created in advance, which associates the discharge speed of stools, the hardness or size of stools, and the classification (for example, hard stools or watery stools) of stools, and thus a correct classification cannot be obtained for a subject that is not set in the table.

It is conceivable to apply the above-mentioned technique of machine learning to analysis of excretion behavior. For example, a learned model is created by executing machine learning through use of learning data that associates features extracted from various images obtained at the time of excretion with a result of classifying or determining those features. It is possible to estimate a desirable analysis result in excretion behavior by inputting, to the learned model, a feature extracted from an image to be analyzed.

When the technique of machines learning is used, a feature is required to be extracted from an image as learning data. Thus, it is necessary to determine how and what kind of features are to be extracted, which costs time for development.

This disclosure provides a determination device, a determination method, and a program capable of reducing time required for development in analysis of excretion behavior using machine learning.

A determination device includes an image information acquirer configured to acquire image information of a subject image obtained by photographing an internal space of a toilet bowl in excretion; an estimator configured to perform estimation regarding a determination matter relating to excretion by inputting the image information to a learned model, the learned model having learned a correspondence relationship between an image for learning and a determination result of the determination matter relating to excretion, the learned model learned by machine learning using a neural network, the image for learning representing an internal space of a toilet bowl in excretion; and a determiner configured to perform determination regarding the determination matter of the subject image based on an estimation result obtained by the estimator.

### BRIEF DESCRIPTION OF THE FIGURES

FIG. 1 is a block diagram illustrating a configuration of a determination system to which a determination device, according to some embodiments;

FIG. 2 is a block diagram illustrating a configuration of a learned model storage, according to some embodiments;

FIG. 3 is a diagram describing an image to be determined by the determination device, according to some embodiments;

FIG. 4 is a flow chart illustrating an overall flow of processing to be executed by the determination device, according to some embodiments;

FIG. 5 is a flow chart illustrating a flow of determination processing to be executed by the determination device, according to some embodiments;

FIG. 6 is a flow chart illustrating a flow of processing of determining a flushing method to be executed by the determination device, according to some embodiments;

FIG. 7 is a diagram describing a determination device, according to some embodiments;

FIG. 8 is a block diagram illustrating a configuration of a determination system to which the determination device, according to some embodiments;

FIG. 9 is a diagram describing processing to be executed by a preprocessor, according to some embodiments;

FIG. 10 is a flow chart illustrating a flow of processing to be executed by the determination device, according to some embodiments;

FIG. 11 is a diagram describing processing to be executed by a preprocessor, according to some embodiments;

FIG. 12 is a flow chart illustrating a flow of processing to be executed by a determination device, according to some embodiments;

FIG. 13 is a diagram describing processing to be executed by a preprocessor, according to some embodiments;

FIG. 14 is a flow chart illustrating a flow of processing to be executed by a determination device, according to some embodiments;

FIG. 15 is a block diagram illustrating a configuration of a determination device, according to some embodiments;

FIG. 16 is a diagram describing processing to be executed by an analyzer, according to some embodiments;

FIG. 17 is a flow chart illustrating a flow of processing to be executed by the determination device, according to some embodiments;

FIG. 18 is a block diagram illustrating a configuration of a learned model storage, according to some embodiments; and

FIG. 19 is a flow chart illustrating a flow of determination processing to be executed by a determination device, according to some embodiments.

### DETAILED DESCRIPTION OF THE DISCLOSURE

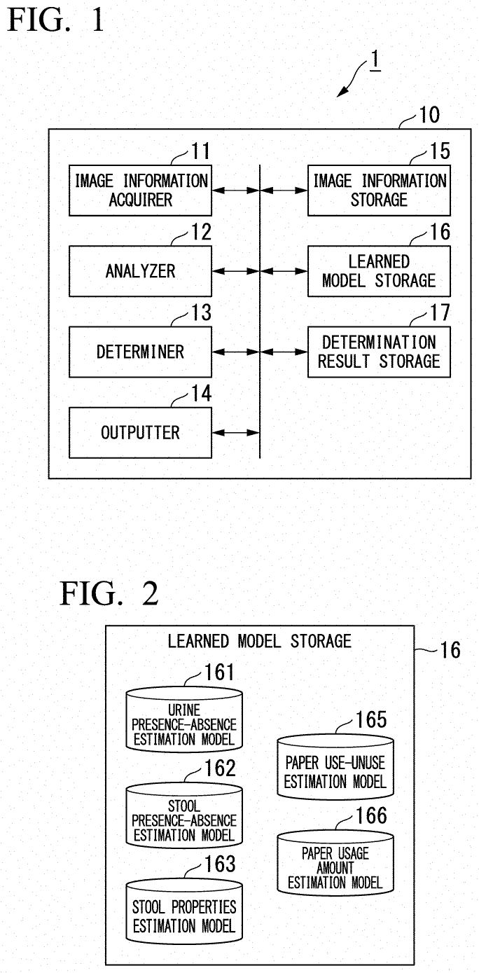

As illustrated in FIG. 1, a determination system 1 includes, for example, a determination device 10.

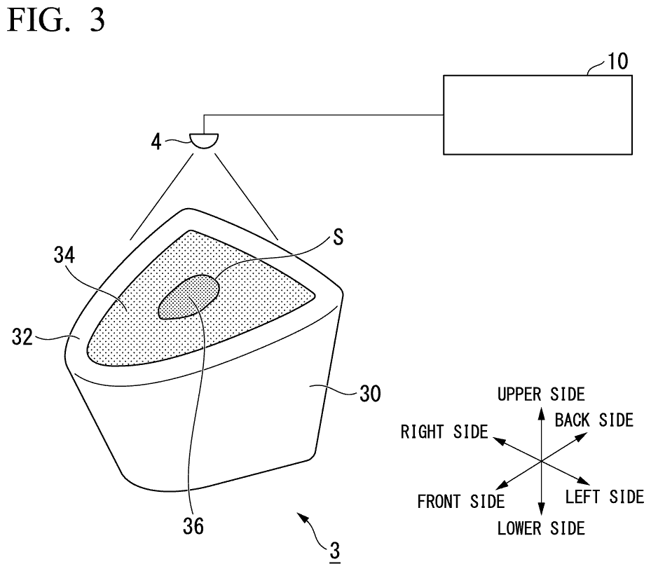

The determination device 10 performs determination relating to excretion based on a subject image (hereinafter also simply referred to as “image”) to be subjected to determination. The subject image is an image relating to excretion, and is, for example, an image obtained by photographing an internal space 34 (refer to FIG. 3) of a toilet bowl 32 (refer to FIG. 3) after excretion. The phrase “after excretion” means any time point after a user has performed excretion and before the toilet is flushed, which is, for example, a time at which the user sitting on the toilet 30 (refer to FIG. 3) has stood up from the toilet 30. Determination relating to excretion means a determination matter relating to the behavior and situation of excretion and flushing of excrement, and includes, for example, presence-absence of excretion, presence-absence of urine, presence-absence of stools, properties of stools, whether or not paper (for example, toilet paper) has been used, a flushing method for the toilet 30 after excretion based on information such as the amount of usage of paper, and the situation of excretion. The properties of stools may be information indicating the state of stools such as “hard stools”, “normal stools”, “soft stools”, “muddy stools”, or “watery stools”, or may be information indicating the properties or state such as “hard” or “soft”. The shape of stools is evaluated by, for example, labeling in terms of, for example, spread on the toilet bowl, how stools are dissolved in a pooled water portion, muddiness, and characteristics in the pooled water (namely, underwater) or the above (namely, in the air) the water surface. The properties of stools may be information indicating the amount of stools, or may be information indicating, for example, two values of whether there is a large amount of stools or a small amount of stools, or three values of whether there is a large amount of stools, a normal amount of stools, or a small amount of stools, or may be information indicating the amount of stools quantitively. The properties of stools may be information indicating the color of stools. The color of stools may be, for example, information indicating whether or not the color of stools is normal under the condition that the color of stools is normal when the color is ocher to brown. In particular, the color of stools may be information indicating whether or not the color of stools is black (color of so-called tarry stools). The flushing method for the toilet 30 after excretion includes, for example, the amount of water and water pressure of flushing water to be used for flushing, and the number of times of flushing.

The determination device 10 includes, for example, an image information acquirer 11, an analyzer 12, a determiner 13, an outputter 14, an image information storage 15, a learned model storage 16, and a determination result storage 17. The analyzer 12 is an example of “estimator”.

The image information acquirer 11 acquires image information on a subject image of the internal space 34 of the toilet bowl 32, which has been photographed in excretion. The image information acquirer 11 outputs the acquired image information to the analyzer 12, and stores the acquired image information into the image information storage 15. The image information acquirer 11 is connected to a toilet device 3 and an image pickup device 4 (refer to FIG. 3).

The analyzer 12 analyzes a subject image corresponding to image information obtained from the image information acquirer 11. Analysis by the analyzer 12 is to estimate the determination matter relating to excretion based on the subject image.

The analyzer 12 performs, for example, estimation by using a learned model that depends on the determination matter of the determiner 13. The learned model is, for example, a model stored in the learned model storage 16, and is a model that has learned a correspondence relationship between a subject image and a result of evaluation relating to excretion.

For example, the analyzer 12 sets, as a result of estimating presence-absence of urine, an output obtained from a learned model that has learned a correspondence relationship between an image and presence-absence of urine. The analyzer 12 sets, as a result of estimating the properties of stools, an output obtained from a learned model that has learned a correspondence relationship between an image and the properties of stools. The analyzer 12 sets, as a result of estimating whether or not paper has been used, an output obtained from a learned model that has learned a correspondence relationship between an image and whether or not paper has been used. The analyzer 12 sets, as a result of estimating the amount of usage of paper, an output obtained from a learned model that has learned a correspondence relationship between an image and the amount of usage of paper.

The analyzer 12 may perform estimation by using a learned model that estimates a plurality of items from an image. For example, the analyzer 12 may perform estimation by using a learned model that has learned a correspondence relationship between an image and presence-absence of urine and stools, respectively. When the learned model has estimated that the image has neither urine nor stools, the analyzer 12 estimates that excretion is not performed.

The determiner 13 performs determination relating to excretion by using an analysis result obtained from the analyzer 12. For example, the determiner 13 sets presence-absence of urine estimated from an image as a determination result of determining presence-absence of urine in that image. The determiner 13 sets presence-absence of stools estimated from an image as a determination result of determining presence-absence of stools in that image. The determiner 13 sets the properties of stools estimated from an image as a determination result of determining the properties of stools in that image. The determiner 13 sets whether or not paper has been used estimated from an image as a determination result of determining whether or not paper has been used in that image. The determiner 13 sets the amount of usage of paper estimated from an image as a determination result of determining the amount of usage of paper in that image.

The determiner 13 may perform determination relating to excretion by using a plurality of estimation results. For example, the determiner 13 may determine the flushing method for the toilet 30 after excretion based on the properties of stools and amount of usage of paper estimated from an image.

The outputter 14 outputs a determination result obtained by the determiner 13. For example, the outputter 14 may transmit the determination result to the terminal of a user who has performed excretion behavior. As a result, the user can recognize his or her excretion behavior and the determination result of the situation. The image information storage 15 stores image information obtained by the image information acquirer 11. The learned model storage 16 stores a learned model corresponding to each of the determination items. The determination result storage 17 stores a determination result obtained by the determiner 13.

The learned model stored in the learned model storage 16 is created by using the technique of deep learning (DL), for example. The DL is a technique of machine learning using a deep neural network (DNN) constructed by multi-layer neural networks. The DNN is implemented by a network created based on the idea of predictive coding in neuroscience, and is constructed by a function configured to simulate a neural circuit. Through use of the technique of DL, it is possible to cause a learned model to automatically recognize a feature inherent in an image in the same way as the cognition of a human. In other words, it is possible to directly perform estimation based on a subject image by causing a learned model to learn data itself of the subject image without performing the task of extracting a feature.

The following description is based on an exemplary case in which the learned model is created by using the technique of DL. However, this disclosure is not limited thereto. The learned model is only required to be a model created by performing learning using learning data that associates image data with the result of evaluating the properties of stools without extracting a feature from the image data. The image data means various images of the internal space 34 of the toilet bowl 32.

As illustrated in FIG. 2, the learned model storage 16 includes, for example, an urine presence-absence estimation model 161, a stool presence-absence estimation model 162, a stool properties estimation model 163, a paper use-unuse estimation model 165, and a paper usage amount estimation model 166.

The urine presence-absence estimation model 161 is a learned model that has learned a correspondence relationship between an image and presence-absence of urine, and is created by performing learning using learning data that associates a subject image with information indicating presence-absence of urine determined from the subject image. The stool presence-absence estimation model 162 is a learned model that has learned a correspondence relationship between an image and presence-absence of stools, and is created by performing learning using learning data that associates a subject image with information indicating presence-absence of stools determined from the subject image.

The stool properties estimation model 163 is a learned model that has learned a correspondence relationship between an image and the properties of stools, and is created by performing learning using learning data that associates a subject image with information indicating the properties of stools determined from the subject image.

The paper use-unuse estimation model 165 is a learned model that has learned a correspondence relationship between an image and whether or not paper has been used, and is created by performing learning using learning data that associates a subject image with information indicating whether or not paper has been used determined from the subject image. The paper usage amount estimation model 166 is a learned model that has learned a correspondence relationship between an image and whether or not paper has been used, and is created by performing learning using learning data that associates a subject image with information indicating the amount of usage of paper determined from the subject image. The amount of usage of paper may be information indicating two values of whether paper usage is large or small, or three values of whether the amount of usage of paper is large, moderate, or small, or may be information indicating the amount of usage of paper quantitively. As the method of determining presence-absence of excretion or the like from an image, for example, it is conceivable that a person in charge of creating learning data determines presence-absence of excretion or the like.

FIG. 3 schematically illustrates a positional relationship between the toilet device 3 and the image pickup device 4.

The toilet device 3 includes, for example, the toilet 30 having the toilet bowl 32. The toilet device 3 is constructed such that flushing water S can be supplied to an opening 36 formed in the internal space 34 of the toilet bowl 32. In the toilet device 3, a functional unit (not shown) provided in the toilet 30 detects, for example, that the user of the toilet device 3 has sat down or stood up, a human's bottom is started to be washed, and an operation of flushing the toilet bowl 32 after excretion is performed. The toilet device 3 transmits the result of detection by the functional unit to the determination device 10.

In the following description, on the assumption that the user of the toilet device 3 has sat on the toilet 30, the front side of the user is referred to as “front side” and the back side of the user is referred to as “back side”. Furthermore, on the assumption that the user of the toilet device 3 has sat on the toilet 30, the left side of the user is referred to as “left side” and the right side of the user is referred to as “right side”. The side away from the floor on which the toilet device 3 is installed is referred to as “upper side”, and the side closer to the floor is referred to as “lower side”.

The image pickup device 4 is provided so as to be capable of picking up an image of details relating to excretion behavior. The image pickup device 4 is installed on the upper side of the toilet 30, for example, the inner side of the edge of the toilet 30 on the back side of the toilet bowl 32 such that the lens of the image pickup device 4 faces the direction of the internal space 34 of the toilet bowl 32. The image pickup device 4 picks up an image in response to an instruction from the determination device 10, for example, and transmits image information of the picked up image to the determination device 10. In this case, the determination device 10 transmits control information indicating an image pickup instruction to the image pickup device 4 via the image information acquirer 11.

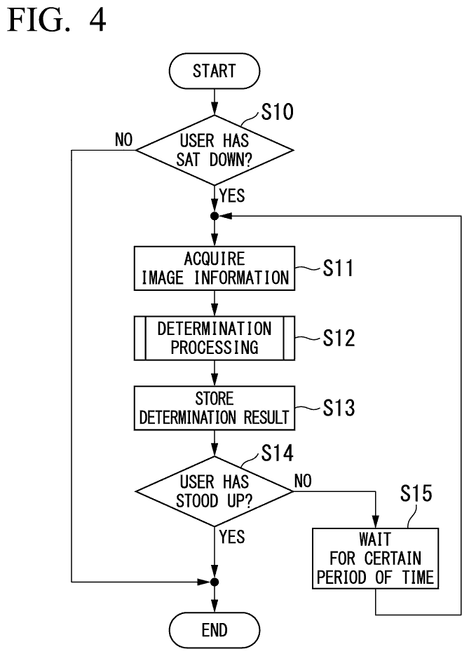

Now, the processing to be executed by the determination device 10 according to some embodiments is described with reference to FIG. 4 to FIG. 6.

An overall flow of the processing to be executed by the determination device 10 is described with reference to FIG. 4. In Step S10, the determination device 10 determines whether or not the user of the toilet device 3 has sat on the toilet 30 through communication with the toilet device 3. When the determination device 10 has determined that the user has sat on the toilet 30, the determination device 10 acquires image information in Step S11. The image information is image information of a subject image. The determination device 10 transmits a control signal instructing the image pickup device 4 to pick up an image, causes the image pickup device 4 to pick up an image of the internal space 34 of the toilet bowl 32, and causes the image pickup device 4 to transmit image information of the picked up image, to thereby acquire the image information. In the flow chart illustrated in FIG. 4, as an example, the determination result of determining that the user has sat is used as a trigger for acquiring the image information. However, this disclosure is not limited thereto. Determination results of other details may be used as the trigger for acquiring the image information. Alternatively, both of the determination result of determining that the user has sat and the results of other details may be used, and when multiple conditions are satisfied, the image information may be acquired. The determination results of other details are, for example, the result of detection by a human detection sensor that detects existence of a person by using, for example, infrared rays. In this case, image acquisition is started when the human detection sensor has detected that the user has approached the toilet 30, for example.

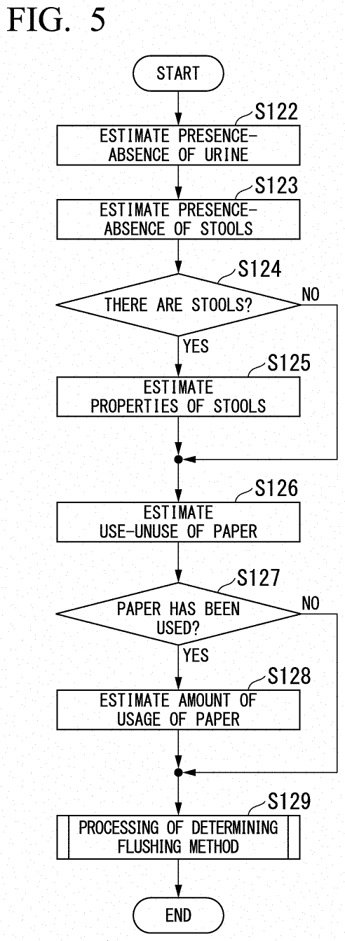

Next, in Step S12, the determination device 10 performs determination processing. Details of the determination processing are described with reference to FIG. 5. In Step S13, the determination device 10 stores the determination result into the determination result storage 17. Next, in Step S14, the determination device 10 determines whether or not the user of the toilet device 3 has stood up from the toilet device 3 through communication with the toilet device 3. When the determination device 10 has determined that the user has stood up, the determination device 10 finishes the processing. On the other hand, when the determination device 10 has determined that the user has not stood up, in Step S15, the determination device 10 waits for a certain period of time, and returns to Step S11.

Now, the flow of determination processing to be executed by the determination device 10 is described with FIG. 5. In Step S122, the determination device 10 uses the urine presence-absence estimation model 161 to estimate presence-absence of urine in the image.

In Step S123, the determination device 10 uses the stool presence-absence estimation model 162 to estimate presence-absence of stools in the image. In Step S124, the determination device 10 determines presence-absence of stools based on the estimation result.

In Step S124, when the determination device 10 has determined that there are stools (YES in Step S124 in FIG. 5), in Step S125, the determination device 10 uses the stool properties estimation model 163 to estimate the properties of stools.

In Step S126, the determination device 10 uses the paper use-unuse estimation model 165 to estimate use-unuse of paper in the image.

In Step S126, when the determination device 10 has estimated that paper has been used (YES in Step S127 in FIG. 5), in Step S128, the determination device 10 uses the paper usage amount estimation model 166 to estimate the amount of usage of paper. In Step S129, the determination device 10 determines the flushing method for the toilet 30 after use of the toilet 30.

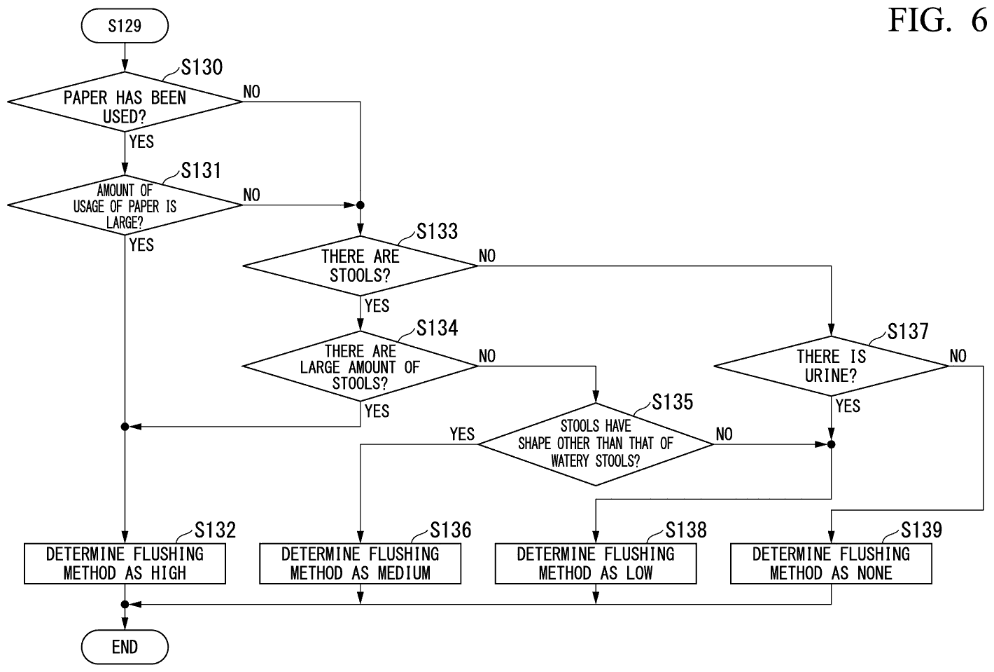

Now, details of the processing of determining the flushing method by the determination device 10 are described with reference to FIG. 6. In the flow chart illustrated in FIG. 6, an exemplary case in which the determination device 10 determines the flushing method as any one of four methods, namely, “high”, “medium”, “low”, and “none” is described. The “high”, “medium”, and “low” in the flushing method mean that the strength of flushing becomes lower in order of “high”, “medium”, and “low”. The strength of flushing means the degree of strength of flushing the toilet bowl 32, and for example, as the strength becomes lower, the amount of flushing water S becomes smaller, whereas as the strength becomes higher, the amount of flushing water S becomes larger. Alternatively, as the strength becomes lower, the number of times of flushing may become smaller, whereas as the strength becomes higher, the number of times of flushing may become larger. When the flushing method is “none”, this means that the toilet bowl 32 is not to be flushed.

In Step S130, the determination device 10 determines use-unuse of paper. When the determination device 10 has determined that paper has been used, in Step S131, the determination device 10 determines whether or not the amount of usage of paper is large. The determination device 10 determines that the amount of usage of paper is large when the amount of paper estimated in Step S126 is equal to or larger than a predetermined threshold value, or determines that the amount of usage of paper is small when the amount of paper estimated in Step S126 is smaller than the predetermined threshold value. When the determination device 10 has determined that the amount of usage of paper is large (YES in Step S131 in FIG. 6), the determination device 10 determines the flushing method as “high” in Step S132.

When the determination device 10 has determined that the amount of usage of paper is small (NO in Step S131 in FIG. 6), the determination device 10 determines whether or not there are stools in Step S133. The determination device 10 determines presence-absence of stools based on the estimation result of presence-absence of stools estimated in Step S123. When the determination device 10 has determined that there are stools (YES in Step S133 in FIG. 6), in Step S134, the determination device 10 determines whether or not there are a large amount of stools. When the amount of stools is equal to or larger than a predetermined threshold value in the properties of stools estimated in Step S125, the determination device 10 determines that there are a large amount of stools, whereas when the amount of stools is smaller than the predetermined threshold value, the determination device 10 determines that there are a small amount of stools. When the determination device 10 has determined that there are a large amount of stools (YES in Step S134 in FIG. 6), the determination device 10 determines the flushing method as “high” in Step S132.

When the determination device 10 has determined that there are a small amount of stools (NO in Step S134 in FIG. 6), in Step S135, the determination device 10 determines whether or not the stools have a shape other than that of watery stools. When the shape of stools is estimated not to be watery stools (that is, the shape of stools is any one of hard stools, normal stools, soft stools, and muddy stools) in the properties of stools estimated in Step S125, the determination device 10 determines that the stools have a shape other than that of watery stools, whereas when the shape of stools is estimated to be watery stools, the determination device 10 determines that the stools have a shape of watery stools. When the determination device 10 has determined that the stools have a shape other than that of watery stools (YES in Step S135 in FIG. 6), the determination device 10 determines the flushing method as “medium” in Step S136. On the other hand, when the determination device 10 has determined that the stools have a shape of watery stools (NO in Step S135 in FIG. 6), the determination device 10 determines the flushing method as “low” in Step S138.

When the determination device 10 has determined that there are no stools in Step S133 (NO in Step S133 in FIG. 6), in Step S137, the determination device 10 determines whether or not there is urine. The determination device 10 determines presence-absence of urine based on the estimation result of presence-absence of urine estimated in Step S122. When the determination device 10 has determined that there is urine (YES in Step S137 in FIG. 6), in Step S138, the determination device 10 determines the flushing method as “low”. On the other hand, when the determination device 10 has determined that there is no urine (NO in Step S137 in FIG. 6), in Step S139, the determination device 10 determines the flushing method as “none”.

As in the example of the flow chart illustrated in FIG. 6, the determination device 10 determines each flushing method based on a combination of results of estimating presence-absence of urine, presence-absence of stools, and presence-absence of paper, to thereby be able to finely control the amount of water for flushing and suppress waste of water to save water appropriately while sufficiently flushing the toilet.

The determination device 10 may determine whether or not the user has performed excretion by using the result of estimating presence-absence of urine illustrated in Step S122 and the result of estimating presence-absence of stools illustrated in Step S123. In this case, the determination device 10 determines that the user has not performed excretion when it is estimated that there is no urine and there is no stools.

As described above, the determination device 10 according to some embodiments includes the image information acquirer 11, the analyzer 12, and the determiner 13. The image information acquirer 11 acquires image information of a subject image obtained by picking up an image of the internal space 34 of the toilet bowl 32. The analyzer 12 inputs the image information into a learned model to estimate the determination matter relating to excretion for the subject image. The determiner 13 performs determination regarding the determination matter of the image based on the estimation result. The learned model is a model that is learned by using the technique of DL. When learning is performed by using the technique of DL, it is only necessary to associate results of determining determination items such as presence-absence of excretion in an image by labeling or the like, and thus learning data is not required to be created by extracting a feature from the image. Thus, there is no need to secure time for considering how and what kind of features are to be extracted. In other words, the determination device 10 according to some embodiments is capable of reducing time required for development in analysis of excretion behavior using machine learning.

In the determination device 10 according some embodiments, the subject image is an image obtained by picking up an image of the internal space 34 of the toilet bowl 32 after excretion. As a result, it is possible to suppress the number of images for determination compared with the case of determining hundreds of images obtained by continuously picking up images of falling excrement, for example. Therefore, it is possible to reduce the load required for estimation or determination, and reduce the time required for development.

In the determination device 10 according to some embodiments, the determination matter include at least any one of presence-absence of urine, presence-absence of stools, and properties of stools. As a result, the determination device 10 according to some embodiments is capable of performing determination relating to excrement.

In the determination device 10 according to some embodiments, the determination matter include use-unuse of paper in excretion and the amount of usage of paper in a case where paper has been used. As a result, the determination device 10 according to some embodiments is capable of performing determination relating to use of paper in excretion, and the determination result can be used for an indicator for determining the flushing method of the toilet 30, for example.

In the determination device 10 according to some embodiments, the determiner 13 determines a flushing method of flushing the toilet 30 under a situation indicated by a subject image. As a result, the determination device 10 according to some embodiments is capable of determining the flushing method for the toilet 30 in addition to determination of excrement.

In the determination device 10 according to some embodiments, the determination matter include at least one of properties of stools and the amount of paper used in excretion, and the analyzer 12 estimates at least one of the properties of stools in a subject image and the amount of paper used in excretion, and the determiner 13 determines the flushing method of flushing the toilet 30 under a situation indicated by the subject image by using the estimation result obtained by the analyzer 12. As a result, the determination device 10 according to some embodiments is capable of determining an appropriate flushing method that depends on excrement or the amount of usage of paper.

In some embodiments, the case of performing determination relating to excrement and performing determination for the flushing method is described as an example. However, determination may be performed only for excrement or the flushing method.

In the determination device 10 according to some embodiments, the determination matter include determination of whether or not excretion has been performed. In this manner, for example, in an elderly facility, when watching over an elderly person, it is possible to grasp whether the elderly person has performed excretion by using the toilet device 3. It is also possible to consider the details of elderly care based on whether or not an elderly person has performed or not performed excretion by himself or herself when the elderly person is guided in a toilet room. The determination result relating to excrement may be used to determine the health condition of a user.

In some embodiments, use-unuse of paper or the like is not determined, and determination is performed only for the properties of stools. In some embodiments, the subject image is subjected to preprocessing. The preprocessing is processing of an image for learning before the model executes machine learning of the image for learning. The preprocessing is processing of an image that is not learned yet before the image that is not learned yet is input to a learned model.

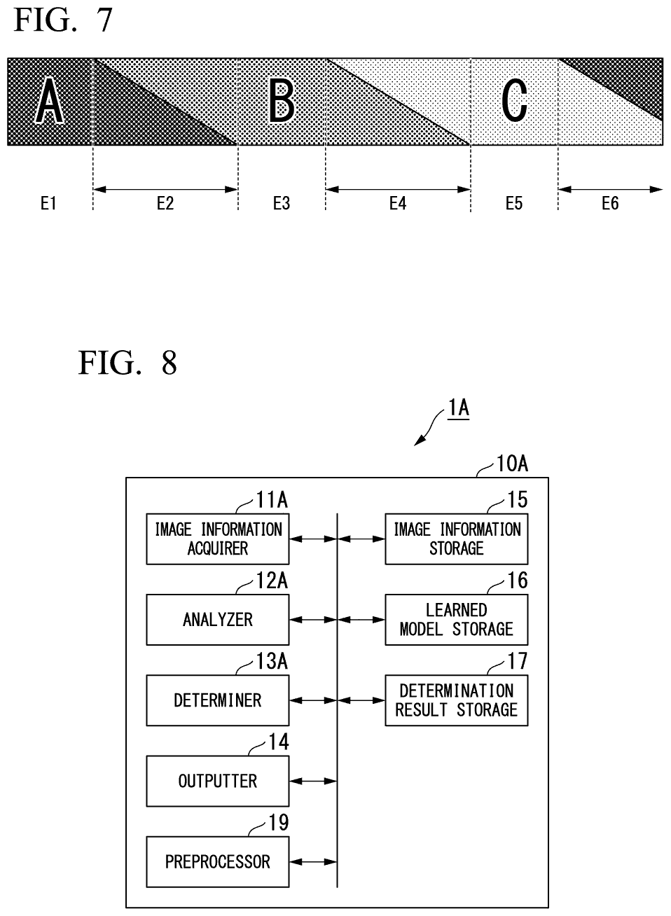

FIG. 7 illustrates a conceptual diagram for describing classification of a specific object into three types A, B, and C. In general, when an object that has a possibility of having various kinds of properties such as stools is classified into three types A, B, and C based on its properties, it is difficult to classify all the objects clearly. In other words, objects of the types A, B, and C are mixed with one another in many cases. For example, as illustrated in FIG. 7, there are a region E1, which can clearly be classified into the type A, a region E2 including the types A and B in a mixed manner, which is classified into the type A or B, a region E3, which can clearly be classified into the type B, a region E4 including the types B and C in a mixed manner, which is classified into the type B or C, a region E5, which can clearly be classified into the type C, and a region E6 including the types C and A in a mixed manner, which is classified into the type C or A.

When the DL is used to construct such a learned model as to classify the properties of stools into the three types A, B, and C, it is considered that the accuracy of estimation deteriorates in a region including the types A, B, and C in a mixed manner. In particular, when watery stools fall into the pooled water surface of the flushing water S pooled in the toilet bowl 32, the fallen watery stools transfer the color of stools to the color of the flushing water S, resulting in diffusion. As a result, even when there are stools with properties different from those of watery stools, which are discharged before the watery stools, there remains little difference in color between the stools with properties different from those of watery stools and the flushing water S having the transferred color. In this case, it is considered that the learned model can no longer recognize the properties of stools with properties different from those of watery stools, and an estimation error occurs. The estimation error is, for example, estimating stools with properties different from those of watery stools to be watery stools even when there are stools with properties different from those of watery stools. When estimation by the learned model has an error, an error occurs in determination of a subject image.

As a countermeasure for this problem, in this embodiment, factors that may cause an estimation error (hereinafter referred to as “noise component”), such as muddiness of the flushing water S, are removed by preprocessing. As a result, it is possible to reduce an estimation error caused by a learned model, and reduce a determination error of a subject image.

As illustrated in FIG. 8, a determination system 1A includes, for example, a determination device 10A. The determination device 10A includes an image information acquirer 11A, an analyzer 12A, a determiner 13A, and a preprocessor 19.

The image information acquirer 11A acquires image information of an image (hereinafter referred to as “reference image”) obtained by picking up an image of the internal space 34 of the toilet bowl 32 before excretion, and image information of a subject image being an image obtained by picking up an image of the internal space 34 of the toilet bowl 32 after excretion. The phrase “before excretion” means any time point before the user of the toilet device 3 performs excretion, and for example, a time point at which the user has entered a toilet room or a time point at which the toilet 30 has sat on the toilet 30.

The preprocessor 19 generates a difference image by using the image information of the reference image and the image information of the subject image. The difference image is an image representing a difference between the reference image and the subject image. The difference means content that is photographed in the subject image but is not photographed in the reference image. In other words, the difference image means an image representing excrement that is photographed in the subject image after excretion but is not photographed in the reference image before excretion.

The preprocessor 19 outputs image information of the generated difference image to the analyzer 12A. The preprocessor 19 may store the image information of the generated difference image into the image information storage 15. The analyzer 12A estimates the properties of stools in the difference image by using a learned model. The learned model to be used for estimation by the analyzer 12A is a model that has learned a correspondence relationship between an image for learning, which represents a difference between images before and after excretion, and a result of evaluating the properties of stools. The image used for learning at the time of creating a learned model, that is, the image for learning, which represents a difference between images before and after excretion, is an example of “difference image for learning”.

The determiner 13A determines the properties of stools shown in the subject image based on the properties of stools estimated by the analyzer 12A. The determiner 13A may determine the situation of excretion of the user based on the properties of stools estimated by the analyzer 12A. The method of determining the situation of excretion of the user by the determiner 13A is described with reference to the flow chart of this embodiment described later.

Now, a method of generating a difference image by the preprocessor 19 is described taking an exemplary case in which the reference image, the subject image, and the difference image are each an RGB image in which the color is represented by R (Red), G (Green), B (Blue). However, the method of representing the color of each image is not limited to RGB, and an image (for example, Lab image or YCbCr image) other than the RGB image can also be generated with a similar method. The RGB value is information indicating the color of an image, and an example of “color information”.

The preprocessor 19 uses a difference between an RGB value of a predetermined pixel in the reference image and an RGB value of a pixel corresponding to the predetermined pixel in the subject image to determine an RGB value of a pixel corresponding to the predetermined pixel in the difference image. The pixel corresponding to the predetermined pixel means a pixel in the same or nearby position coordinates in the image. The difference indicates a difference in color between two pixels, and is determined based on a difference between RGB values, for example. For example, the preprocessor 19 determines that there is no difference when the RGB values indicate the same color, or determines that there is a difference when the RGB values do not indicate the same color.

For example, when the RGB value of a predetermined pixel in the reference image is (255, 255, 0) (that is, yellow) and the RGB value of a predetermined pixel in the subject image is (255, 255, 0) (that is, yellow), there is no difference in color between the two pixels, and thus mask processing of setting the RGB value of a predetermined pixel in the difference image to a predetermined color (for example, white) indicating no difference is executed.

When the RGB value of a predetermined pixel in the reference image is (255, 255, 0) (that is, yellow) and the RGB value of a predetermined pixel in the subject image is (255, 0, 0) (that is, red), there is a difference in color between the two pixels, and thus the RGB value of a predetermined pixel in the difference image is set to the RGB value (255, 0, 0) (that is, red) of the predetermined pixel in the subject image.

When there is a difference in color between two pixels, the preprocessor 19 may set the RGB value of a predetermined pixel in the difference image to a predetermined color (for example, black) indicating a difference.

When there is a difference in color between two pixels, the preprocessor 19 may set the RGB value of a predetermined pixel in the difference image to a predetermined color depending on the degree of difference. The degree of difference is a value calculated depending on a vector distance between RGB values in a color space, for example. In this case, the preprocessor 19 classifies the difference in color between two pixels into a plurality of values depending on the degree of difference. For example, when the degree of difference is classified into three types of values, namely, “large”, “medium”, and “small”, the preprocessor 19 may generate a difference image by setting the RGB value of a pixel having a large degree of difference in the difference image to black, setting the RGB value of a pixel having a medium degree of difference in the difference image to gray, and setting the RGB value of a pixel having a small degree of difference in the difference image to light gray, for example.

The amount of light to be radiated to the internal space 34 of the toilet bowl 32 being a subject is considered to change due to an influence of the degree of sitting by the user or the like. When the amount of light has changed, the strength of color of a portion with no change before and after excretion changes in some cases. In such a case, the preprocessor 19 is considered to determine a change in thickness of color as a difference in color.

As a countermeasure for this problem, the preprocessor 19 may determine the color of a predetermined pixel in the difference image depending on the ratio of the color of a predetermined pixel in the reference image and the ratio of the color of a predetermined pixel in the subject image. The ratio of the color is the ratio of each color of RGB, and is indicated by a proportion with respect to a predetermined reference value, for example. Specifically, the ratio of the color of the RGB value (R, G, B) is R/L:G/L:B/L. L represents a predetermined reference value. The predetermined reference value L may be any value. The predetermined reference value L may be a value that is fixed irrespective of the RGB value, or may be a value (for example, R value of RGB value) that changes depending on the RGB value.

For example, when a predetermined pixel in the reference image is gray (that is, RGB value (128, 128, 128)) and a predetermined pixel in the subject image is light gray (that is, RGB value (192, 192, 192)), the ratios of the colors of the two pixels are the same, and thus the preprocessor 19 determines that there is no difference in color between the two pixels.

When a predetermined pixel in the reference image is yellow (that is, RGB value (255, 255, 0)) and a predetermined pixel in the subject image is red (that is, RGB value (255, 0, 0)), the ratios of the colors of the two pixels are not the same, and thus the preprocessor 19 determines that there is a difference in color between the two pixels.

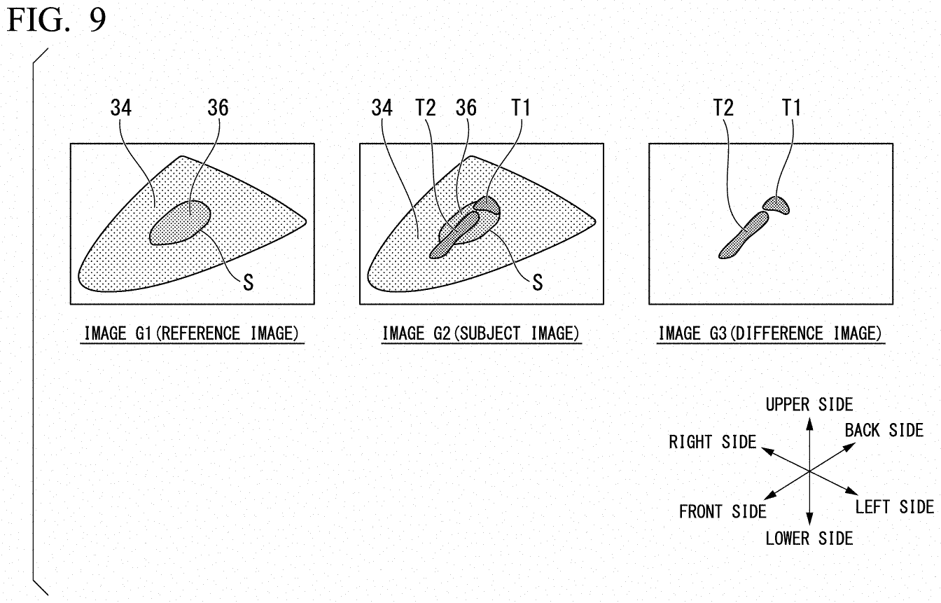

The left side of FIG. 9 represents an image G1 as an example of the reference image, the center of FIG. 9 represents an image G2 as an example of the subject image, and the right side of FIG. 9 represents an image G3 as an example of the difference image. As illustrated in the image G1 of FIG. 9, the internal space 34 before excretion is photographed in the reference image, and a situation in which the flushing water S is stored in the opening 36 substantially at the center of the internal space 34 is photographed. As illustrated in the image G2 of FIG. 9, the internal space 34 after excretion is photographed in the subject image, and a situation in which there are excrements T1 and T2 on the upper side of the flushing water S in the directions of the front side and back side of the internal space 34. As illustrated in the image G3 of FIG. 9, the excrements T1 and T2, which are differences between the reference image and the subject image, are represented in the difference image.

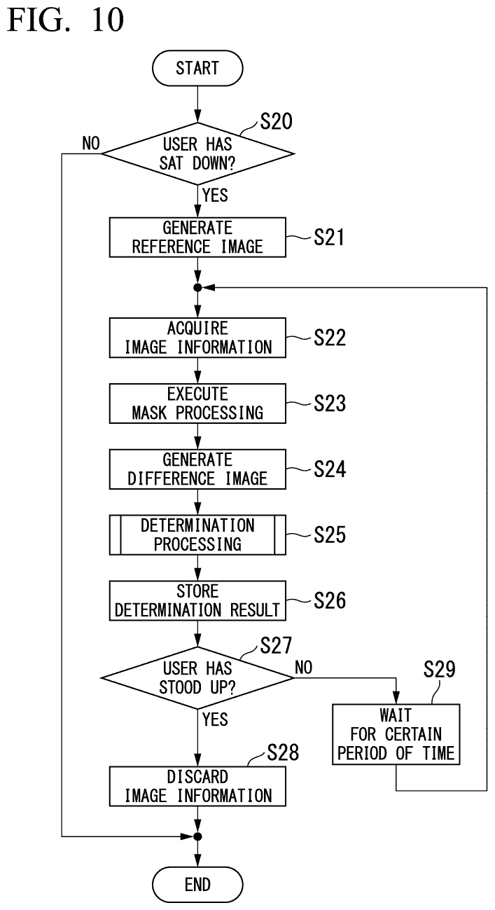

Now, the processing to be executed by the determination device 10A according to some embodiments is described with reference to FIG. 10. In the flow chart illustrated in FIG. 10, Step S20, Step S22, Step S25 to Step S27, and Step S29 are similar to Step S10, Step S11, Step S12 to Step S14, and Step S15 of the flow chart of FIG. 4, and thus description thereof is omitted here.

In Step S21, when the determination device 10A has determined that the user has sat on the toilet 30, the determination device 10A generates a reference image. The reference image is an image representing the internal space 34 of the toilet bowl 32 before excretion. When the determination device 10A has determined that the user has sat on the toilet 30, the determination device 10A transmits a control signal instructing the image pickup device 4 to pick up an image, to thereby acquire image information of the reference image.

In Step S23, the determination device 10A performs mask processing by using the reference image and the subject image. The mask processing is processing of setting a pixel with no difference between the reference image and the subject image to a predetermined color (for example, white). In Step S24, the determination device 10A generates a difference image. The difference image is, for example, an image obtained by executing mask processing for the pixel with no difference between the reference image and the subject image, and reflecting a pixel value of the subject image, namely, an RGB value, for the pixel with a difference between the reference image and the subject image.

In Step S28, when the determination device 10A has determined that the user has stood up from the toilet 30, the determination device 10A discards image information of the reference image, the subject image, and the difference image. Specifically, the determination device 10A deletes image information of the reference image, the subject image, and the difference image, which has been stored in the image information storage 15. As a result, it is possible to suppress excessive use of the storage capacity.

As described above, the determination processing illustrated in Step S25 of FIG. 10 is similar to the processing illustrated in Step S12 of FIG. 4. However, in this embodiment, at least the determination processing configured to set the properties of stools as the determination item is only required to be executed.

In Step S25 of FIG. 10, the determiner 13A determines the situation of excretion of the user by using the result of estimating the properties of stools in the difference image. For example, when the shape of stools is hard stools, the determiner 13A determines that the situation of excretion of the user is likely to be constipation. When the shape of stools is normal stools, the determiner 13A determines that the situation of excretion of the user is good. When the shape of stools is soft stools, the determiner 13A determines that the situation of excretion of the user is follow-up required. When the shape of stools is muddy stools or watery stools, the determiner 13A determines that the situation of excretion of the user is likely to be diarrhea. The determiner 13A may determine the health condition of the user based on the situation of excretion.

As described above, in the determination device 10A according to the second embodiment, the preprocessor 19 generates a difference image indicating a difference between the reference image and the subject image. As a result, the determination device 10A according to some embodiments is capable of showing a portion with a difference before and after excretion in the difference image, and thus it is possible to grasp the properties of excrement more accurately and determine the properties more accurately.

In the determination device 10A according to some embodiments, the preprocessor 19 uses a difference between color information indicating a color of a predetermined pixel in the reference image and color information of a pixel corresponding to the predetermined pixel among pixels of the subject image to determine color information of a pixel corresponding to the predetermined pixel in the difference image. As a result, the determination device 10A according to some embodiments is capable of showing a portion with a difference in color before and after excretion in the difference image, and thus it is possible to exhibit an effect similar to that of the above-mentioned effect.

In the determination device 10A according to some embodiments, the preprocessor 19 sets a difference between an RGB value of a predetermined pixel in the reference image and an RGB value of a pixel corresponding to the predetermined pixel in the subject image as an RGB value of a pixel corresponding to the predetermined pixel in the difference image. As a result, the determination device 10A according to some embodiments is capable of recognizing a difference in color before and after excretion as a difference between RGB values. Thus, it is possible to calculate the difference in color quantitively, and exhibit an effect similar to that of the above-mentioned effect.

In the determination device 10A according to some embodiments, the preprocessor 19 uses a difference between the color ratio indicating the ratio of the R value, the G value, and the B value of a predetermined pixel in the reference image and the color ratio of a pixel corresponding to the predetermined pixel in the subject image to determine an RGB value of a pixel corresponding to the predetermined pixel in the difference image. As a result, even when a difference in background color has occurred due to, for example, a difference in amount of light radiated to a subject before and after excretion, the determination device 10A according to some embodiments is capable of extracting the properties of excrement without erroneously recognizing the difference as excrement, and exhibiting an effect similar to that of the above-mentioned effect.

In the description given above, the image information acquirer 11A acquires image information of the reference image as an example. However, this disclosure is not limited thereto. For example, image information of the reference image may be acquired by any functional unit, or may be stored in the image information storage 15 in advance.

Modification example 1, in some embodiments, includes a divided image, which is obtained by dividing the subject image, is generated as preprocessing. In the following description, the configuration equivalent to those of embodiments described above is assigned with the same reference numeral, and description thereof is omitted here.

In general, the toilet bowl 32 is formed so as to be inclined toward the lower side from the edge of the toilet bowl 32 toward the opening 36. Thus, when there are a plurality of stools that have fallen into the toilet bowl 32, it is considered that a stool that has fallen first is pushed by a stool that has fallen next to move toward the lower side of the toilet bowl 32 along the inclined surface thereof. In other words, the toilet bowl 32 has such a characteristic that a stool that has fallen first moves toward the front side of the opening 36.

In this modification example, estimation that considers discharge of excrement in time series is performed by using this characteristic. Specifically, the subject image is divided into the front side and the back side. Then, the properties of stools are determined by considering excrement photographed in an image (hereinafter referred to as “front-side divided image”) obtained by extracting the front side of the subject image as old stools, and considering excrement photographed in an image (hereinafter referred to as “back-side divided image”) obtained by extracting the back side of the subject image as new stools. As a result, regarding the situation of excretion of the user, it is possible to perform determination based on stools closer to the current state by determining old stools.

In this modification example, the preprocessor 19 generates a divided image. The divided image is an image including a partial region of the subject image, and is, for example, a front-side divided image or a back-side divided image. The boundary for dividing the subject image into the front-side divided image and the back-side divided image may be set in any manner. For example, the subject image is divided into the front-side divided image and the back-side divided image by a line in a left-right direction (that is, direction connecting between left side and right side) passing through the center of the pooled water surface of the flushing water S pooled in the toilet bowl 32.

The divided image is not limited to the front-side divided image and the back-side divided image described above. The divided image is only required to be an image including at least a partial region of the subject image. The subject image may be divided into three regions in the front-back direction (that is, direction connecting between front side and back side), or the front-side divided image may be further divided into a plurality of regions in the left-right direction. One divided image or a plurality of divided images may be generated from the subject image. When a plurality of divided images are generated from the subject image, a region combining the regions represented by the plurality of divided images may be the entire region or partial region of the subject image.

The preprocessor 19 outputs image information of the generated divided image to the analyzer 12A. The preprocessor 19 may generate the image information of the generated divided image into the image information storage 15. The analyzer 12A estimates the properties of stools in the divided image by using a learned model. The learned model to be used for estimation by the analyzer 12A is a model that has learned a correspondence relationship between an image for learning, which is obtained by dividing an image obtained by photographing the internal space 34 of the toilet bowl 32 in excretion, and a result of evaluating the properties of stools.

The determiner 13A determines the situation of excretion of the user under a situation indicated by the subject image based on the properties of stools in the divided image estimated by the analyzer 12A. When there are a plurality of divided images generated from the subject image, the determiner 13A determines the situation of excretion of the user by considering the estimation result for each divided image in an integrated manner. The method of determining the situation of excretion of the user in an integrated manner by the determiner 13A is described with reference to the flow chart of this modification example described later.

Now, description is given of an image to be used for learning at the time of creating a learned model, that is, an image for learning, which is obtained by dividing an image obtained by photographing the internal space 34 of the toilet bowl 32 in excretion. The divided image serving as an image for learning in this modification example is an example of “divided image for learning”. The divided image serving as an image for learning is an image obtained by extracting a partial region of various images of the internal space 34 of the toilet bowl 32, which are photographed at the time of past excretion. The method of dividing an image by a preprocessor 23 may be any method, but is desired to be a method similar to a method of dividing an image by the preprocessor 19. By using a similar method, improvement in accuracy of estimation using a learned model can be expected. It is possible to set the learned model to be a model that estimates the state of a region more accurately because the learned model is caused to learn a partial region of the subject image, that is, a region narrower than the subject image compared with the case of learning the entire subject image.

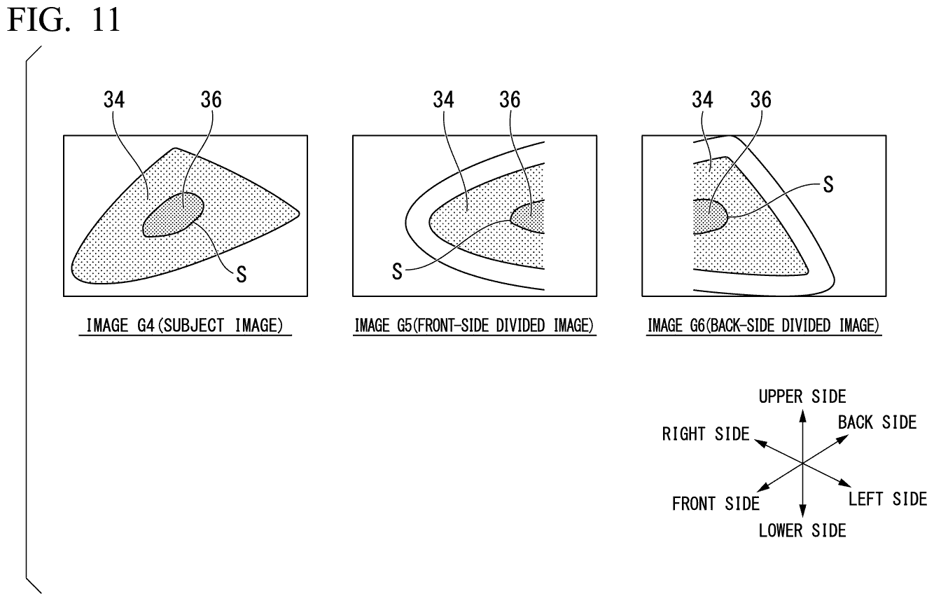

The left side of FIG. 11 represents an image G4 as an example of the reference image, the center of FIG. 11 represents an image G5 as an example of the front-side divided image, and the right side of FIG. 11 represents an image G6 as an example of the back-side divided image. As illustrated in the image G4 of FIG. 11, the internal space 34 is photographed in the subject image, and the entire internal space 34 is photographed, which includes a situation in which the flushing water S is stored in the opening 36 substantially at the center of the internal space 34. As illustrated in the image G5 of FIG. 11, a region on the front side of the internal space 34 is extracted in the front-side divided image, and specifically, a region on the front side with respect to a boundary line in the left-right direction passing through the center of the pooled water surface of the opening 36 storing the flushing water S is extracted. As illustrated in the image G6 of FIG. 11, a region on the back side of the internal space 34 is extracted in the back-side divided image, and specifically, a region on the back side with respect to the boundary line in the left-right direction passing through the center of the pooled water surface is extracted.

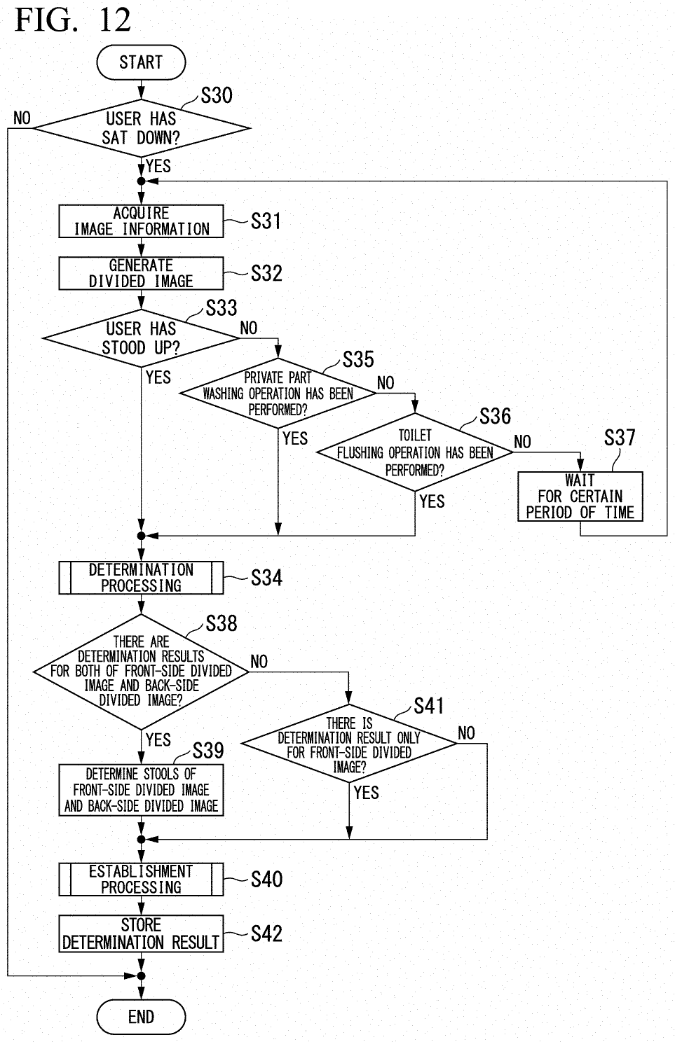

Now, the processing to be executed by the determination device 10A according to the modification example 1 of some embodiments is described with reference to FIG. 12. FIG. 12 is a flow chart illustrating a flow of processing to be executed by the determination device 10A according to the modification example 1 of the second embodiment. In the flow chart illustrated in FIG. 12, Step S30, Step S31, Step S33, Step S37, and Step S42 are similar to Step S10, Step S11, Step S14, Step S15, and Step S13 of the flow chart of FIG. 4, and thus description thereof is omitted here.

In Step S32, the determination device 10A generates a divided image by using a subject image. The divided image is, for example, a front-side divided image representing a region on the front side of a region photographed in the subject image, and a back-side divided image representing a region on the back side of the region photographed in the subject image.

In Step S34, the determination device 10A performs determination processing for each of the front-side divided image and the back-side divided image. Details of this determination processing are similar to those of the processing illustrated in Step S25 in the flow chart of FIG. 10, and thus description thereof is omitted here.

In Step S35, when the determination device 10A has not determined that the user has stood up from the toilet 30 (NO in Step S33 in FIG. 12), the determination device 10A determines whether or not a human's bottom washing operation in the toilet 30 has been performed, and when a human's bottom washing operation in the toilet 30 has been performed, the determination device 10A performs the processing illustrated in Step S34. In Step S36, when the determination device 10A has not determined that a human's bottom washing operation in the toilet 30 has been performed (NO in Step S35 in FIG. 12), the determination device 10A determines whether or not a toilet flushing operation in the toilet 30 has been performed, and when a toilet flushing operation in the toilet 30 has been performed, the determination device 10A determines performs the processing illustrated in Step S34.

In Step S38, the determination device 10A determines whether or not there are determination results for both of the front-side divided image and the back-side divided image. The phrase “there are determination results for both of the front-side divided image and the back-side divided image” means that both of the front-side divided image and the back-side divided image each include an image of stools, and have a determination result for the properties of the image of stools. In Step S39, when there are determination results for both of the front-side divided image and the back-side divided image, the determination device 10A sets the determination result for the front-side divided image as a determination result of old stools, and sets the determination result for the back-side divided image as a determination result of new stools.

In Step S40, the determination device 10A performs establishment processing by the determiner 13A. The establishment processing is processing of establishing the situation of excretion of the user by using the determination result of new stools and the determination result of old stools. The determination device 10A establishes the situation of excretion by considering that the old stools represent the current situation of excretion, for example. In the establishment processing, for example, when the properties of old stools are determined to be hard stools and the properties of new stools are determined to be normal stools, the determiner 13A determines that hard stools in the large intestine have been discharged at the time of excretion, and the situation of excretion of the user is likely to be constipation. On the other hand, in the establishment processing, for example, when the properties of old stools are determined to be normal stools and the properties of new stools are determined to be muddy stools, the determiner 13A determines that the situation of excretion of the user is good.

In Step S41, when there is a determination result for only one of the front-side divided image and the back-side divided image, the determiner 13A of the determination device 10A determines whether or not there is a determination result for the front-side divided image. When there is a determination result for the front-side divided image, the determiner 13A performs the processing illustrated in Step S40 by using the determination result for the front-side divided image. On the other hand, when there is no determination result for the front-side divided image, the determiner 13A performs the processing illustrated in Step S40 by using the determination result for the back-side divided image. The phrase “when there is no determination result for the front-side divided image” means, for example, a case in which excrement is not photographed in the front-side divided image and the properties of stools have failed to be determined.

As described above, in the determination device 10A according to the modification example 1 of the second embodiment, the preprocessor 19 generates a divided image including a partial region of a subject image. As a result, the determination device 10A according to the modification example 1 of some embodiments is capable of specifically determining a partial region of a subject image, which enables a narrow region to be determined in detail and achieves more accurate determination compared with the case of determining the entire subject image.

In the determination device 10A according to the modification example 1 of the second embodiment, the preprocessor 19 generates a front-side divided image representing at least a region on the front side of the toilet bowl in the subject image. As a result, when new stools and old stools are photographed in the subject image, the determination device 10A according to the modification example 1 of some embodiments is capable of setting, as a divided image, a region in which the new stools are considered to be photographed. Even when new stools and old stools are not photographed in the subject image, the determination device 10A according to the modification example 1 of some embodiments is capable of setting, as a divided image, a region in which stools are likely to be photographed, which achieves an effect similar to that of the above-mentioned effect.

In the determination device 10A according to the modification example 1 of some embodiments, the preprocessor 19 generates a front-side divided image and a back-side divided image, the analyzer 12A performs estimation regarding a determination matter of the front-side divided image and performs estimation regarding the determination matter of the back-side divided image, and the determiner 13A performs determination regarding the determination matter of the subject image by using an estimation result for the front-side divided image and an estimation result for the back-side divided image. As a result, the determination device 10A according to the modification example 1 of some embodiments is capable of determining the situation of excretion of the user in an integrated manner by using the estimation results for the front-side divided image and the back-side divided image, and achieving accurate determination compared with the case of using any one of the estimation results for the front-side divided image and the back-side divided image.

In the determination device 10A according to the modification example 1 of some embodiments, the preprocessor 19 sets an estimation result for a front-side divided image as an estimation result older than an estimation result for a back-side divided image, and performs determination regarding the determination matter of the subject image. As a result, the determination device 10A according to the modification example 1 of some embodiments is capable of performing determination that considers excretion in time series by considering the estimation result for the front-side divided image as an estimation result of old stools and considering the estimation result for the back-side divided image as an estimation result of new stools, to thereby achieve accurate determination closer to the current state for the situation of excretion of the user. The direction of movement of stools that have fallen first changes depending on the shape of the toilet bowl 32, and thus a temporal relationship between the front-side divided image and the back-side divided image may be opposite. Specifically, in the description given above, the front-side divided image is set to be older than the back-side divided image. However, this disclosure is not limited thereto, and the front-side divided image may be considered to be newer than the back-side divided image to perform the determination and establishment processing.

Modification example 2 can include entire image representing the entire subject image and a partial image obtained by extracting a part of the subject image are generated as preprocessing. In the following description, the configuration equivalent to those of embodiments described above is assigned with the same reference numeral, and description thereof is omitted here.

In general, when a machine learning technique is used to estimate specific determination details based on the entire image, high calculation capabilities are required, which increases costs of devices. For example, when the number of layers in a DNN used as a model is increased, the number of times of calculation required for one trial increases due to increase in number of nodes, resulting in increase of processing loads. In order for the model to estimate specific details, that is, to minimize an error between output of a model in response to input of learning data and an output in the learning data, it is necessary to perform trials repeatedly while changing a weight W and a bias component b. In order to cause such repeated trials to converge within a realistic period, a device capable of processing a large amount of calculations at high speed is required. In other words, a high-performance device is required to analyze the entire subject image in detail, which increases costs of devices.

The subject image is an image obtained by photographing the entire internal space 34 of the toilet bowl 32. In other words, the subject image includes a region in which excrement is photographed and a region in which excrement is not photographed. Thus, it is conceivable to adopt a method of extracting, from the subject image, a specific region (for example, region near the opening 36) in which excrement is likely to fall, and estimate specific determination details for the extracted region. As a result, it is possible to reduce the region of an image to be analyzed, and suppress increase in costs of devices.

In the first place, it is not clear where excrement is likely to fall in the toilet bowl 32. The properties of stools change depending on the physical condition of the user. Thus, even when the region in which excrement falls is a specific region in the toilet bowl 32 in many cases, excrement does not always fall into the specific region, and excrement may be scattered around the specific region. When determination is performed by using only the image of a specific region without using an image representing the surroundings of the specific region regardless of the fact that excrement is scattered around the specific region, the result of determination may be different from the actual situation.

As a countermeasure for this problem, in this modification example, an entire image representing the entire subject image and a partial image obtained by extracting a part of the subject image are generated by preprocessing.

The entire image is used to perform comprehensive determination, which is not specific, to suppress increase in costs of devices. The phrase “comprehensive determination” means determination that is more overall and comprehensive than determination of the properties of stools, and for example, means determining presence-absence of scattered stools. Presence-absence of scattered stools can be determined relatively roughly and easily compared with the case of determining the properties of stools because the properties of scattered stools are not determined. Determination of presence-absence of scattered stools, which is performed for the entire image, is an example of “first determination matter”.

Determination of a specific determination item, which is more specific than determination for the entire image, is performed for a partial image. The specific determination item means, for example, determination of the properties of stools. The specific determination item is determined for a partial image, which is obtained by reducing the region of an image to be determined, to thereby be capable of performing specific determination and suppressing the cost of devices without using a high-performance device. Determination of the properties of stools, which is performed for a partial image, is an example of “second determination matter”.

In this modification example, the preprocessor 19 generates an entire image and a partial image. The entire image is an image representing the entire subject image, and for example, is a subject image itself. The partial image is an image obtained by extracting a partial region of the subject image, and is, for example, an image obtained by extracting a nearby region of the opening 36 from the subject image. Which part of region is to be extracted from the subject image as the partial image may be set in any manner, and for example, a fixed region determined at the time of shipment or the like depending on the shape of the toilet 30 may be extracted.

The preprocessor 19 outputs the generated entire image and partial image to the analyzer 12A. The preprocessor 19 may store image information on the generated entire image and partial image into the image information storage 15.

The analyzer 12A uses a learned model to estimate presence-absence of scattered stools in the entire image. Estimation of presence-absence of scattered stools in the entire image is an example of “first estimation”.

The analyzer 12A uses a learned model to estimate the properties of stools in the partial image. The processing of estimating the properties of stools in the partial image is an example of “second estimation”.

The determiner 13A determines the situation of excretion of the user under a situation indicated by the subject image based on presence-absence of scattered stools in the entire image estimated by the analyzer 12A and the properties of stools in the partial image. The method of determining the situation of excretion of the user by the determiner 13A based on the estimation result for the entire image and the estimation result for the partial image is described later with reference to the flow chart of this modification example.

Now, learning data to be learned by the learned model used in this modification example is described. The learned model to be used for estimation for the entire image is a model that has learned a correspondence relationship between the entire image for learning, which is obtained by photographing the entire internal space 34 of the toilet bowl 32 in excretion, and an evaluation result of evaluating presence-absence of scattered stools. The entire image for learning means various kinds of images representing the entire internal space 34 of the toilet bowl 32, which was photographed in the past at the time of excretion. The entire image for learning, that is, an image for learning, which is obtained by photographing the entire internal space 34 of the toilet bowl 32 in excretion, is an example of “entire image for learning”. The learned model to be used for estimating the partial image is a model that has learned a correspondence relationship between a partial image for learning, which is obtained by extracting a part of an image of the entire internal space 34 of the toilet bowl 32 in excretion, and an evaluation result of evaluating the properties of stools. The partial image for learning is an image obtained by extracting a part of the entire image. The partial image for learning, that is, an image for learning, which is obtained by extracting a part of an image of the entire internal space 34 of the toilet bowl 32 in excretion is an example of “partial image for learning”. The method of generating the entire image for learning and the partial image for learning may be any method, but is desired to be a method similar to the method of generating the entire image and the partial image by the preprocessor 19. Improvement in accuracy of estimation using a learned model is expected by using a similar method.

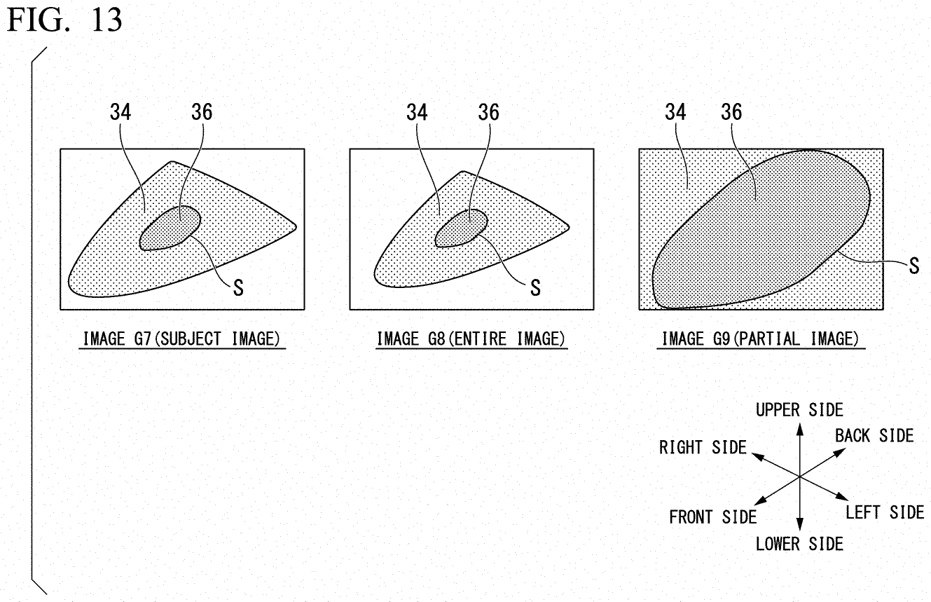

FIG. 13 is a diagram describing processing to be executed by the preprocessor 19 according to the modification example 2. The left side of FIG. 13 represents an image G7 as an example of the subject image, the center of FIG. 11 represents an image G8 as an example of the entire image, and the right side of FIG. 11 represents an image G9 as an example of the partial image. As illustrated in the image G7 of FIG. 13, the internal space 34 is photographed in the subject image, and the entire internal space 34, which includes the situation in which the flushing water S is stored in the opening 36 substantially at the center of the internal space 34. As illustrated in the image G8 of FIG. 13, the entire subject image is illustrated in the entire image. The entire image may be the subject image itself, or the entire image may be an image obtained by extracting the entire subject image. As illustrated in the image G9 of FIG. 13, a nearby region of the opening 36 substantially at the center of the internal space 34 is extracted in the partial image, and the pooled water surface of the flushing water S and a region near the pooled water surface are extracted.

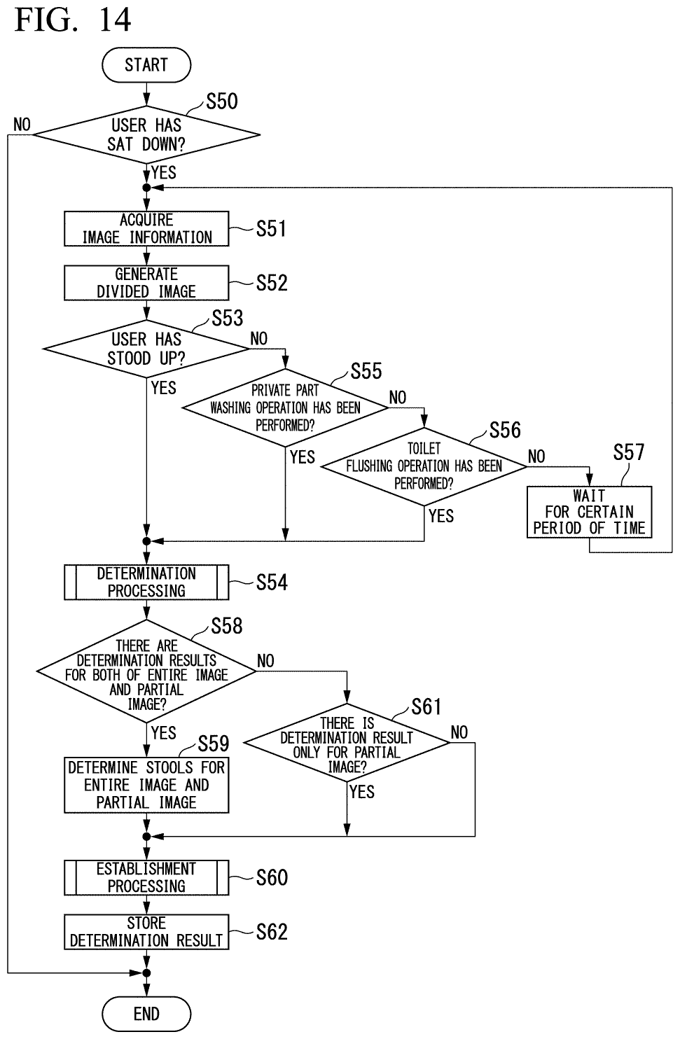

Now, the processing to be executed by the determination device 10A according to the modification example 2 of some embodiments is described with reference to FIG. 14. FIG. 14 is a flow chart illustrating a flow of processing to be executed by the determination device 10A according to the modification example 2. In the flow chart illustrated in FIG. 14, Step S50, Step S51, Step S53, Step S57, and Step S62 are similar to Step S10, Step S11, Step S14, Step S15, and Step S13 of the flow chart of FIG. 4, and thus description thereof is omitted here. In the flow chart illustrated in FIG. 14, Step S55 and Step S56 are similar to Step S35 and Step S36 of the flow chart of FIG. 12, and thus description thereof is omitted here.

In Step S52, the determination device 10A generates an entire image and a partial image by using a subject image. The entire image is, for example, an image representing the entire region photographed in the subject image. The partial image is, for example, an image representing a specific partial region photographed in the subject image.

In Step S54, the determination device 10A performs determination processing for each of the entire image and the partial image. The determination device 10A performs comprehensive determination for the entire image, for example, determination of presence-absence of scattered stools. The determination device 10A estimates presence-absence of scattered stools in the entire image by using a learned model, and sets the estimated result as a determination result of determining presence-absence of scattered stools in the entire image. The learned model is a model created by performing learning using learning data that associates the entire image for learning with the determination result of determining presence-absence of scattered stools. The determination device 10A performs specific determination for the partial image, for example, determination of the properties of stools. The determination device 10A estimates the properties of stools in the partial image by using a learned model, and sets the estimated result as a determination result of determining the properties of stools in the partial image. The learned model is a model created by performing learning using learning data that associates the partial image for learning with the determination result of determining the properties of stools.

In Step S58, the determination device 10A determines whether there are determination results for both of the entire image and the partial image. The phrase “there are determination results for both of the entire image and the partial image” means that presence-absence of scattered stools in the entire image is determined and the properties of stools are determined for the partial image.

In Step S59, when the determination device 10A has determined that there are determination results for both of the entire image and the partial image, which are obtained by the determiner 13A, the determination device 10A corrects the determination result for the partial image by using the determination result for the entire image. Correcting the determination result for the partial image means changing or correcting the determination result for the partial image by using the determination result for the entire image. For example, when the determiner 13A has determined that there are scattered stools based on the determination result for the entire image in a case where the properties of stools are determined to be soft stools based on the determination result for the partial image, the determiner 13A corrects the situation of excretion such that the situation of excretion is likely to be diarrhea. On the other hand, when the determiner 13A has determined that there are no scattered stools based on the determination result for the entire image, the determiner 13A does not correct the situation of excretion serving as the determination result for the partial image.

In Step S60, the determination device 10A performs establishment processing by the determiner 13A. The establishment processing is processing determining the situation of excretion of the user or the like by using the determination result for the entire image and the determination result for the partial image.

In Step S61, when the determination device 10A has determined that there are not determination results for both of the entire image and the partial image, which are obtained by the determiner 13A, the determination device 10A determines whether or not there is a determination result for the partial image. When there is a determination result for the partial image, the determination result for the partial image is used to perform processing illustrated in Step S60. When there is no determination result for the partial image, the determination result for the entire image is used to perform processing illustrated in Step S60. The phrase “there is no determination result for the partial image” means, for example, a case in which excrement is not photographed in the partial image and the properties of stools have failed to be determined.

As described above, in the determination device 10A according to the modification example 2 of the second embodiment, the preprocessor 19 generates an entire image and a partial image from a subject image. The analyzer 12A performs first estimation, which is comprehensive estimation, based on the entire image by using a learned model, and performs second estimation, which is specific estimation, based on the partial image by using another learned model. As a result, the determination device 10A according to the modification example 2 of some embodiments performs relatively easy comprehensive estimation by using an entire image having a large number of pixels, to thereby be capable of reducing the load of calculation processing and suppressing increase in cost of devices compared with the case of performing relatively difficult specific estimation based on the entire image. It is possible to reduce the load of calculation processing and suppressing increase in cost of devices compared with the case of performing specific estimation based on an entire image having a relatively large number of pixels by performing specific estimation using a partial image having a relatively small number of pixels.

In the determination device 10A according to the modification example 2 of some embodiments, the preprocessor 19 generates a partial image including at least the opening 36 of the toilet bowl 32 in the subject image. As a result, the determination device 10A according to the modification example 2 is capable of extracting a region into which excrement is likely to fall, and performing specific estimation relating to excrement by using the partial image.

In the determination device 10A according to the modification example 2 of some embodiments, the preprocessor 19 estimates presence-absence of scattered stools as comprehensive estimation (that is, first estimation), and estimates the properties of stools as specific estimation (that is, second estimation). As a result, the determination device 10A according to the modification example 2 of some embodiments is capable of estimating presence-absence of scattering as well as the properties of stools, and performing determination more accurately by using both the estimation results.

The determination device 10A according to the modification example 2 corrects the estimation result of specific estimation (that is, second estimation) by using the estimation result of comprehensive estimation (that is, first estimation). As a result, the determination device 10A according to the modification example 2 is capable of correcting specific estimation and performing determination more accurately.

In some embodiments, a determination region in a subject image is extracted. The determination region is a region for which determination is performed in this embodiment, which is a region for which the properties of excrement are determined. In other words, the determination region is a region in which excrement is estimated to be photographed in the subject image. In the following description, the configuration equivalent to those of embodiments described above is assigned with the same reference numeral, and description thereof is omitted here.

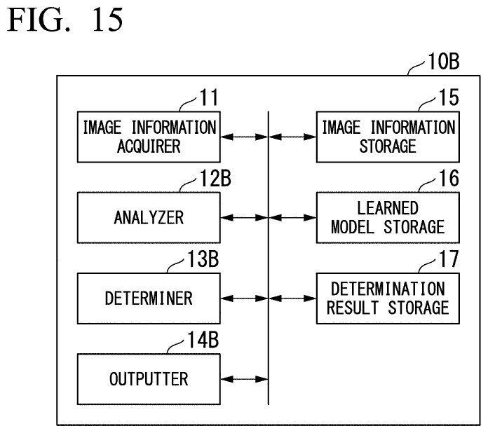

As illustrated in FIG. 15, the determination device 10B includes an analyzer 12B and a determiner 13B. The analyzer 12B is an example of “extractor”.

The analyzer 12B uses a difference between the color of the subject image and a predetermined color (hereinafter referred to as “expected color”), that is, a color difference, to extract a region with a color close to the expected color as a determination region. The analyzer 12B determines whether or not the color of the subject image is a color close to the expected color based on a distance (hereinafter referred to as “spatial distance”) between both the colors in a color space. When the spatial distance between the two colors is small, this means that the color difference is small and the two colors are close to each other. On the other hand, when the spatial distance is large, this means that the color difference is large, and the two colors are away from each other. The spatial distance is an example of “characteristic of expected color”.

Now, a method of calculating a spatial distance by the analyzer 12B is described. In the following description, the subject image is an RGB image and the expected color is a color indicated by the RGB value as an example. However, this disclosure is not limited thereto. The determination region can be extracted by a similar method also when the subject image is an image (for example, Lab image or YCbCr image) other than an RGB image, or when the expected color is indicated by a color (for example, Lab image or YCbCr image) other than an RGB value. In the following description, the expected color is the color of stools as an example. However, this disclosure is not limited thereto. The expected color is only required to be a color that excrement is expected to have, and may be, for example, the color of urine.

The analyzer 12B calculates, for example, a Euclidean distance in the color space as the spatial distance. The analyzer 12B calculates the Euclidean distance in accordance with Expression (1) given below. In Expression (1), Z1 represents an Euclidean distance, ΔR represents a difference between an R value of a predetermined pixel X in the subject image and an R value of an expected color Y, ΔG represents a difference between a G value of the pixel X and a G value of the expected color Y, and AB represents a difference between a B value of the pixel X and a B value of the expected color Y. The RGB value of the predetermined pixel X in the subject image is (red, green, blue), and the RGB value of the expected color Y is (Rs, Gs, Bs).

$$Z 1 = ( Δ R \hat{} 2 + Δ G \hat{} 2 + Δ B \hat{} 2 ) \hat{} ( 1 / 2 ) \qquad ( 1 )$$

where ΔR=red-Rs, ΔG=green-Gs, and ΔB=blue-Bs

The analyzer 12B may add a weight when calculating the spatial distance. A weight is added to emphasize a difference in specific component forming a color. For example, a weight is added by multiplying an R component, a G component, and a B component, which form a color, by different weight coefficients, respectively. It is possible to emphasize a color difference with an expected color depending on the component by adding a weight.

The analyzer 12B can calculate a weighted Euclidean distance in accordance with Expression (2) given below, for example. In Expression (2), Z2 represents a weighted Euclidean distance, R_COEF represents a weight coefficient of the R component, G_COEF represents a weight coefficient of the G component, and B_COEF represents a weight coefficient of the B component. AR represents a difference between an R value of the pixel X and an R value of the expected color Y, ΔG represents a difference between a G value of the pixel X and a G value of the expected color Y, and AB represents a difference between a B value of the pixel X and a B value of the expected color Y. The RGB value of the predetermined pixel X in the subject image is (red, green, blue), and the RGB value of the expected color Y is (Rs, Gs, Bs).

$$Z 2 = ( R\_COEF \times Δ R \hat{} 2 + G\_COEF \times Δ G \hat{} 2 + B\_COEF \times Δ B \hat{} 2 ) \hat{} ( 1 / 2 ) \qquad ( 2 )$$

where R_COEF>G_COEF>B_COEF, ΔR=red-Rs, ΔG=green-Gs, and ΔB=blue-Bs

The R component tends to have a stronger characteristic of the color of stools, which is the expected color Y, than the G component, and the G component tends to have a stronger characteristic of the color of stools than the B component. The analyzer 12B sets the weight coefficient of the R component to be larger than the weight coefficient of the G component based on the characteristic of each component forming such a color. That is, in Expression (2), the relationship of R_COEF>G_COEF>B_COEF is satisfied for the coefficient R_COFE, the coefficient G_COFE, and the coefficient B_COFE.

The amount of light to be radiated to the internal space 34 of the toilet bowl 32, which is a subject, is considered to change due to an influence of the degree of sitting by the user or the like. When the amount of light has changed, pieces of excrement with the same color may be photographed such that the thicknesses of color are different. In such a case, even when pieces of excrement have the same color, the spatial distances of the pieces of excrement are calculated to be different distances.

As a countermeasure for this problem, the analyzer 12B may calculate, as the spatial distance, the Euclidean distance of a ratio (hereinafter referred to as “color ratio”) in each component forming the color. For example, the color ratio is obtained by dividing the value of one component by the value of another component serving as a reference among the R value, the G value, and the B value. Through use of the color ratio, it is possible to calculate the spatial distance in which the difference due to the thickness of color is not reflected.

The component serving as a reference at the time of deriving the color ratio may be determined in any manner, and for example, it is conceivable to set a component dominant in that color as the reference. For example, the R component is dominant in the color of stools. Thus, in this embodiment, the color ratio is created by dividing each of the R value, the G value, and the B value by the R value.

For example, the color ratio of the pixel X (RGB value (red, green, blue)) is (red/red, green/red, blue/red), that is, (1, green/red, blue/red). The color ratio of the expected color Y (RGB value (Rs, Gs, Bs)) is (Rs/Rs, Gs/Rs, Bs/Rs), that is, (1, Gs/Rs, Bs/Rs).

The analyzer 12B can calculate the Euclidean distance of the color ratio in accordance with Expression (3) given below. In Expression (3), Z3 represents the Euclidean distance of the color ratio, ΔRp represents a difference between the R component of the color ratio of the pixel X and the R component of the color ratio of the expected color Y, ΔGp represents a difference between the G component of the color ratio of the pixel X and the G component of the color ratio of the expected color Y, and ΔBp represents a difference between the B component of the color ratio of the pixel X and the B component of the color ratio of the expected color Y. GR_RATE represents the ratio of the G component in the color ratio of the expected color Y, and BR_RATE represents the ratio of the B component in the color ratio of the expected color Y. The RGB value of a predetermined pixel X in the subject image is (red, green, blue), and the RGB value of the expected color Y is (Rs, Gs, Bs).

$$Z 3 = ( Δ Rp \hat{} 2 + Δ Gp \hat{} 2 + Δ Bp \hat{} 2 ) \hat{} ( 1 / 2 ) = ( Δ Gp \hat{} 2 + Δ Bp \hat{} 2 ) \hat{} ( 1 / 2 ) \qquad ( 3 )$$

where ΔRp=red/red-Rs/Rs=0 (zero), ΔGp=green/red-GR_RATE, ΔBp=blue/red-BR_RATE, GR_RATE=Gs/Rs, BR_RATE=Bs/Rs, and 1>GR_RATE>BR_RATE>0

The R component tends to have a stronger characteristic of the color of stools, which is the expected color Y, than the G component (that is, Rs>Gs), and the G component tends to have a stronger characteristic of the color of stools than the B component (that is, Gs>Bs). The ratio GR_RATE and the ratio BR_RATE are both values that fall within a range of from 0 (zero) to 1. The value of BR_RATE is smaller than that of GR_RATE. That is, in Expression (3), the relationship of 1>GR_RATE>BR_RATE>0 is satisfied for the ratio GR_RATE and the ratio BR_RATE.

The analyzer 12B may add a weight to a specific component forming the color ratio when calculating the Euclidean distance of the color ratio. The analyzer 12B can calculate the Euclidean distance that has weighted the color ratio in accordance with Expression (4) given below. In Expression (4), Z4 represents a Euclidean distance that has weighted the color ratio. ΔRp represents a difference between the R value of the pixel X and the R value of the expected color Y, ΔGp represents a difference between the G value of the pixel X and the G value of the expected color Y, ΔBp represents a difference between the B value of the pixel X and the B value of the expected color Y. GR_COEF represents the weight coefficient of the difference ΔGp, and BR_COEF represents the weight coefficient of the difference ΔBp. The RGB value in the predetermined pixel in the subject image is (red, green, blue), and the RGB value in the expected color Y is (Rs, Gs, Bs).

$$Z 4 = ( GR\_COEF \times Δ Gp \hat{} 2 + BR\_COEF \times Δ Bp \hat{} 2 ) \hat{} ( 1 / 2 ) \qquad ( 4 )$$

where GP_COEF>BP_COEF, ΔGp=green/red-GR_RATE, ΔBp=blue/red-BR_RATE, GR_RATE=Gs/Rs, BR_RATE=Bs/Rs, and 1>GR_RATE>BR_RATE>0

In Expression (4), the relationship of GP_COEF>BP_COEF is satisfied for the coefficient GR_COFE and the coefficient BR_COFE similarly to the relationship of the coefficient G_COFE and the coefficient B_COFE in Expression (2). In Expression (4), the relationship of 1>GR_RATE>BR_RATE>0 is satisfied for the ratio GR_RATE and the ratio BR_RATE similarly to Expression (3). For example, the ratio GR_RATE=0.7, the ratio BR_RATE=0.3, the coefficient GR_COFE=40, and the coefficient BR_COFE=1 are set.

The analyzer 12B creates an image obtained by gray scaling the spatial distance calculated for each pixel of the subject image (hereinafter referred to as “gray scale subject image”). For example, the analyzer 12B uses Expression (5) to adjust the scale of the spatial distance and obtain a converted gray scale value. In Expression (5), Val represents a gray scale value, ΔMP represents a coefficient for adjusting the scale, and Z represents the spatial distance. The spatial distance Z may be any one of a Euclidean distance Z1 of the RGB value, a weighted Euclidean distance Z2 in the RGB value, a Euclidean distance Z3 in the color ratio, and a weighted Euclidean distance Z4 in the color ratio. Z_MAX represents a maximum value of the spatial distance calculated for each pixel of the subject image, and Val_MAX represents a maximum value of the gray scale value.

$$Val = AMP \times Z \qquad ( 5 )$$

where AMP=Val_MAX/Z_MAX

For example, when the gradation of the gray scale is represented by 256 values, namely, 0 to 255, in the gray scale subject image, the maximum value Val_MAX of the gray scale value is 255. In this case, the spatial distance Z is converted into the gray scale value Val so that the maximum value X_MAX of the spatial distance is the maximum value Val_MAX(255) of the gray scale by using Expression (5). As a result, the analyzer 12B creates a gray scale subject image that represents the spatial distance to the expected color by the gray scale value of from 0 (that is, white) to 255 (that is, black).

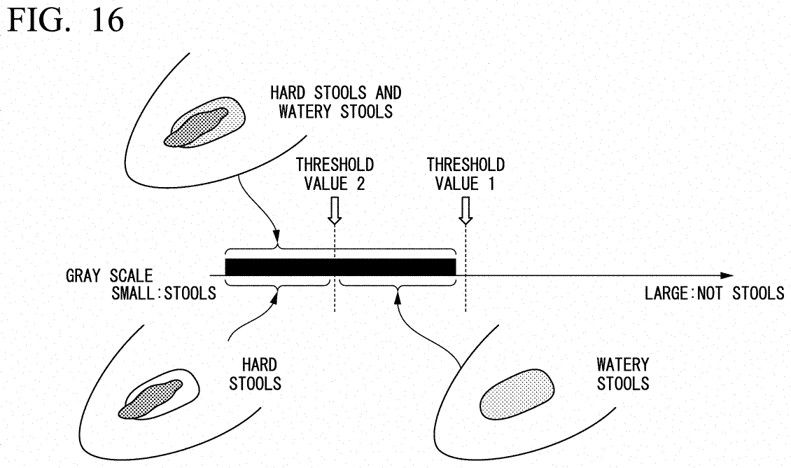

Now, a method of extracting a determination region by the analyzer 12B is described with reference to FIG. 16. FIG. 16 is a diagram describing processing to be executed by the analyzer 12B according to the third embodiment. In FIG. 16, a gray scale axis is set in the left-right direction, which indicates that the gray scale value increases as a value on the gray scale axis moves from the left side toward the right side.

As illustrated in FIG. 16, in the gray scale subject image, a pixel having a small spatial distance is represented by a small gray scale value. That is, a color closer to the color of stools, which is the expected color, is represented by a small gray scale value, and a region having a small gray scale value can be considered to be a region in which stools are photographed. On the other hand, a pixel having a large spatial distance is represented by a large gray scale value in the gray scale subject image. That is, a color that is away from the color of stools, which is the expected value, is represented by a large gray scale value, and a region having a large gray scale value can be considered to be a “non-stools” region in which stools are not photographed.

The analyzer 12B extracts a determination region by using this characteristic. Specifically, the analyzer 12B determines, as a region including excrement, a region for which the gray scale value of a pixel in the gray scale subject image is smaller than a predetermined first threshold value (hereinafter also referred to as “threshold 1”), and extracts the region including excrement as a determination region. The first threshold value is a gray scale value that corresponds to a boundary that distinguishes between the color of the flushing water S pooled in the toilet bowl 32 and the color of watery stools.

When the color of hard stools and the color of watery stools are compared with each other, watery stools are dissolved in the flushing water S, and thus the color of watery stools is considered to be lighter than that of hard stools. In this case, the gray scale value corresponding to the color of watery stools is represented by darker gray than the gray scale value corresponding to the color of hard stools, which indicates that the color of watery stools is away from the color of stools, which is the expected color.

Through use of this characteristic, the analyzer 12B extracts the region of watery stools and the region of hard stools in a distinguished manner from the determination region. Specifically, the analyzer 12B determines, as a region of hard stools, a region for which the gray scale value is smaller than a predetermined second threshold value (hereinafter also referred to as “threshold value 2”), and determines, as a region of watery stools, a region for which the gray scale value is equal to or larger than the second threshold value within the determination region of the gray scale subject image. The second threshold value is set to be a value smaller than the first threshold value. The region of watery stools is an example of “determination region”. The region of hard stools is an example of “determination region”.

When the determination region includes the region of watery stools and the region of hard stools in a mixed manner, the analyzer 12B may extract the two regions (the region of watery stools and the region of hard stools) in a distinguished manner. When the determination region includes two regions, the range of the data scale that may be taken by a pixel included in the determination region is a combination of the range of the gray scale that may be taken by watery stools and the range of the gray scale that may be taken by hard stools, resulting in a relatively wide range. On the other hand, when the determination region includes only one region (that is, region of watery stools or region of hard stools), the range of the data scale that may be taken by a pixel included in the determination region is a relatively narrow range.

Through use of this characteristic, the analyzer 12B determines whether or not the determination region includes the region of watery stools and the region of hard stools in a mixed manner depending on the range of the gray scale in a pixel included in the determination region. For example, the analyzer 12B sets, as the range of the gray scale, a difference between the maximum value and the minimum value of the gray scale in a pixel included in the determination region. When the range of the gray scale in the determination region is smaller than a predetermined difference threshold value, the analyzer 12B determines that the determination region does not include the region of watery stools and the region of hard stools in a mixed manner, that is, determines that the determination region includes only the region of watery stools or the region of hard stools. When the range of the gray scale in the determination region is equal to or larger than the predetermined difference threshold value, the analyzer 12B determines that the determination region includes the region of watery stools and the region of hard stools in a mixed manner. Through use of the range of the gray scale that may be taken by watery stools and the range of the gray scale that may be taken by hard stools, the difference threshold value is set to, for example, a wider range, a narrower range, and a value corresponding to a representative value of the two ranges. The representative value may be any one of generally used representative values such as a simple average, a weighted average, and a median of the two ranges.

The analyzer 12B outputs, to the determiner 13B, information of an image (hereinafter referred to as “extracted image”) representing the extracted determination region. In this case, when the determination region includes the region of watery stools and the region of hard stools in a mixed manner, the analyzer 12B outputs, to the determiner 13B, information of an image (hereinafter referred to as “watery portion extracted image”) representing the region of watery stools in the determination region and an image (hereinafter referred to as “hard portion extracted image”) representing the region of hard stools in the determination region. On the other hand, when the determination region does not include the region of watery stools and the region of hard stools in a mixed manner, the analyzer 12B outputs, to the determiner 13B, information of an image (hereinafter referred to as “watery portion extracted image”) representing the region of watery stools in the determination region and an image (hereinafter referred to as “hard portion extracted image”) representing the region of hard stools in the determination region.

Referring back to FIG. 15, the determiner 13B performs determination regarding a determination matter based on the extracted image acquired by the analyzer 12B. Specifically, the determiner 13B uses the watery stools extracted image to determine the properties of watery stools. The determiner 13B uses the hard stools extracted image to determine the properties of hard stools. The determiner 13B uses the watery portion extracted image to determine the properties of watery stools. The determiner 13B uses the hard portion extracted image to determine the properties of hard stools.

Similarly to the other embodiments described above, the determiner 13B may determine the properties of stools by using an estimation result obtained by machine learning. In this case, the analyzer 12B may perform estimation by machine learning, or other functional units may perform estimation. The determiner 13B may determine the properties of stools by using other image analysis techniques. In this case, the determination device 10B can omit the learned model storage 16.

The determiner 13B is not required to analyze the entire subject image because the determination region is extracted by the analyzer 12B and the determiner 13B can analyze the narrow image. The determiner 13B can analyze an image in which watery stools and hard stools are distinguished from each other, and thus it becomes easy to perform the processing of determining the properties compared with the case of analyzing an image in which watery stools and hard stools are not distinguished from each other.

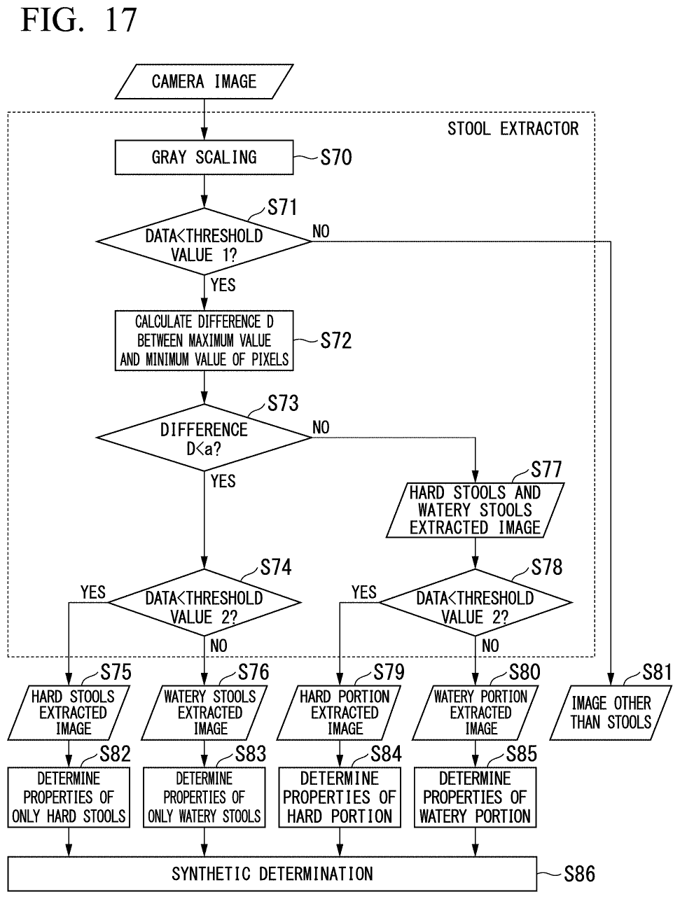

Now, the processing to be executed by the determination device 10B is described with reference to FIG. 17. This flow chart illustrates the flow of processing after the processing of acquiring image information is performed. The processing of acquiring image information is the processing corresponding to Step S11 of the flow chart illustrated in FIG. 4, which corresponds to processing described as “camera image” in this flow chart.

In Step S70, the analyzer 12B creates a gray scale subject image by gray scaling the subject image. In Step S71, the analyzer 12B determines whether or not the gray scale value of each pixel in the gray scale subject image is smaller than the first threshold value. In Step S72, the analyzer 12B calculates a difference D between the maximum value and the minimum value of the gray scale values for a group of pixels of the determination region for which the gray scale value is smaller than the first threshold value in the gray scale subject image.

In Step S73, the analyzer 12B determines whether or not the difference D is smaller than a difference threshold value a. When the difference D is smaller than the difference threshold value a, the analyzer 12B determines that the determination region does not include the region of watery stools and the region of hard stools in a mixed manner, and proceeds to the processing illustrated Step S74. In Step S74, the analyzer 12B determines whether or not the gray scale value of each pixel in the determination region is smaller than the second threshold value. When the gray scale value of each pixel in the determination region is smaller than the second threshold value, in Step S75, the analyzer 12B outputs the hard stools extracted image to the determiner 13B. In Step S82, the determiner 13B determines the properties of stools (hereinafter referred to as “hard stools focused stools”) focused on hard stools based on the hard stools extracted image. When the gray scale value of each pixel in the determination region is equal to or larger than the second threshold value, in Step S76, the analyzer 12B outputs the watery stools extracted image to the determiner 13B. In Step S83, the determiner 13B determines the properties of stools (hereinafter referred to as “watery stools focused stools”) focused on watery stools based on the watery stools extracted image.

When the difference D is equal to or larger than the predetermined difference threshold value a (NO in Step S73 in FIG. 17), in Step S77, the analyzer 12B determines that the determination region includes the region of watery stools and the region of hard stools in a mixed manner. In Step S78, the analyzer 12B determines whether or not the gray scale value of each pixel in the determination region is smaller than the second threshold value. When the gray scale value of each pixel in the determination region is smaller than the second threshold value, in Step S79, the analyzer 12B outputs the region to the determiner 13B as a hard portion extracted image. In Step S84, the determiner 13B determines the properties of stools in the hard portion, which is the region of hard stools in a mixed state, based on the hard portion extracted image. When the gray scale value of each pixel in the determination region is equal to or larger than the second threshold value, in Step S76, the analyzer 12B outputs the region to the to the determiner 13B as a watery portion extracted image. In Step S85, the determiner 13B determines the properties of stools in the watery portion, which is the region of watery stools in a mixed state, based on the watery portion extracted image.

In Step S86, the determiner 13B uses the result of determining the properties of stools in Step S82 to Step S85 to determine the properties of stools in the subject image in an integrated manner.

In Step S81, the determiner 13B determines that the image is an image other than stools and excludes the image from the determination region for a group of pixels for which the gray scale value of each pixel in the gray scale subject image is equal to or larger than the first threshold value (threshold value 1) in Step S71.

As described above, in the determination device 10B according to the third embodiment, the analyzer 12B extracts a determination region from the subject image based on the characteristic of the expected color Y. As a result, the determination device 10B is capable of extracting a region including excrement from the subject image. A region for determining the properties of stools can be narrowed down, and thus it is possible to reduce the processing load required for determination compared with the case of analyzing the entire subject image. A device that does not have high calculation capabilities can perform processing by reducing the processing load, and thus it is possible to suppress increase in device cost. The determination region can be extracted based on the characteristic of the expected color Y of excrement to be determined, and thus it becomes easy to perform determination in the determination region compared with a region extracted irrespective of the expected color Y.

In the determination device 10B according to the third embodiment, the analyzer 12B calculates the spatial distance Z to the expected color Y in the color space for the color of each pixel in the subject image, and extracts a set of pixels for which the calculated spatial distance Z is smaller than a predetermined threshold value as the determination region. As a result, the determination device 10B according to some embodiments is capable of calculating a difference in color with the expected color Y, that is, the color difference by using the spatial distance Z, determining a region having a small color difference with the expected color Y, and extracting a determination region based on a quantitative indicator.

In the determination device 10B according to some embodiments, the analyzer 12B calculates, for the color of each pixel in the subject image, the spatial distance in the color space by using a value obtained by adding a weight to a difference for each component of the expected color Y. As a result, the determination device 10B according to some embodiments is capable of calculating a spatial distance emphasizing a component (for example, R component) that is likely to produce a difference with the expected color Y. In this manner, it is possible to extract a determination region accurately.

In the determination device 10B according to some embodiments, the subject image is an RGB image, the expected color is a color indicated by the RGB value, and the analyzer 12B calculates a spatial distance in the color space by using the color ratio indicating the ratio of the R value, the G value, and the B value of each pixel in the subject image, and values obtained by adding weights to a difference between the ratio of the R component and the color ratio of the expected color Y, a difference between the ratio of the G component and the color ratio of the expected color Y, and a difference between the ratio of the B component and the color ratio of the expected color Y. As a result, the determination device 10B is capable of calculating a spatial distance without being influenced by a difference in thickness of color due to a difference in amount of light radiated to a subject. In this manner, it is possible to extract a determination region accurately.

In the determination device 10B, the analyzer 12B creates a gray scale subject image, sets a region for which the gray scale value of a pixel in the gray scale subject image is smaller than a predetermined first threshold value as a region including excrement, and extracts the region including excrement as the determination region. As a result, the determination device 10B according to some embodiments is capable of extracting a determination region by a simple method of comparing the gray scale value of each pixel in the gray scale subject image with a threshold value.

In the determination device 10B, the analyzer 12B sets, as a region representing watery stools, a region for which the gray scale value of a pixel in the gray scale subject image is smaller than the first threshold value and is equal to or larger than a predetermined second threshold value smaller than the first threshold value, sets, as a region representing hard stools, a region for which the gray scale value of a pixel in the gray scale subject image is smaller than the second threshold value, and extracts, as the determination region, the region representing watery stools and the region representing hard stools. As a result, the determination device 10B according to some embodiments is capable of extracting a determination region by distinguishing between the region representing watery stools and the region representing hard stools by a simple method of comparing the gray scale value of each pixel in the gray scale subject image with a threshold value, and extracting a determination region accurately. It is possible to reduce the processing load of determination by the determiner 13B by distinguishing between the region representing watery stools and the region representing hard stools, and extracting a determination region compared with the case of not distinguishing between the region representing watery stools and the region representing hard stools.

In the description given above, the analyzer 12B uses one gray scale subject image to extract a determination region as an example. However, this disclosure is not limited thereto. The analyzer 12B may extract a determination region by using a plurality of different gray scale subject images. For example, the analyzer 12B may perform only the processing of extracting a determination region based on the first threshold value by using a gray scale subject image obtained by converting the Euclidean distance Z4, which has weighted the color ratio, into the gray scale. The analyzer 12B may perform only the processing of distinguishing between the region representing watery stools and the region representing hard stools based on the second threshold value by using a gray scale subject image obtained by converting the Euclidean distance Z1 into the gray scale.

In the description given above, a plurality of embodiments have been described. However, the configuration of each embodiment is not limited to the embodiment, and may be used for the configurations of other embodiments. For example, the difference image, the divided image, the entire image, and the partial image may be used for the processing of determining the properties of stools. The gray scale subject image may be used for the difference image or the like. The difference image, the divided image, the entire image, and the partial image may be used for the processing of determining the properties of stools.

The determination device 10C determines whether or not dirt due to an image pickup device or an image pickup environment is photographed in a subject image. Dirt due to an image pickup device or an image pickup environment is shadow, stain, or the like different from a subject, which is photographed in a subject image. For example, dirt due to an image pickup device or an image pickup environment is, for example, filth, urine, dirty water, or the like attached to, for example, a lens due to scattering of excrement because excrement has been discharged or excrement has fallen into the toilet bowl 32. In other cases, dirt due to an image pickup device or an image pickup environment is water droplets attached to, for example, a lens at the time of flushing the toilet. In other cases, dirt due to an image pickup device or an image pickup environment is water droplets attached to, for example, a lens at the time of human's bottom washing that outputs wash water from a nozzle. Fingerprints or the like attached to, for example, a lens are also an example of “dirt due to an image pickup device or an image pickup environment”.

In the following description, whether or not a lens of an image pickup device is dirty (hereinafter also referred to as “lens dirt”) is determined as an example. However, this disclosure is not limited thereto. For example, when photography is performed under a state in which a waterproof plate is mounted on the outside of the lens of the image pickup device, whether or not the waterproof plate is dirty is determined. Lens dirt is an example of “dirt due to an image pickup device or an image pickup environment”. The dirt of a waterproof plate in a case where the waterproof plate is mounted on the outside of the lens of the image pickup device is an example of “dirt due to an image pickup device or an image pickup environment”.

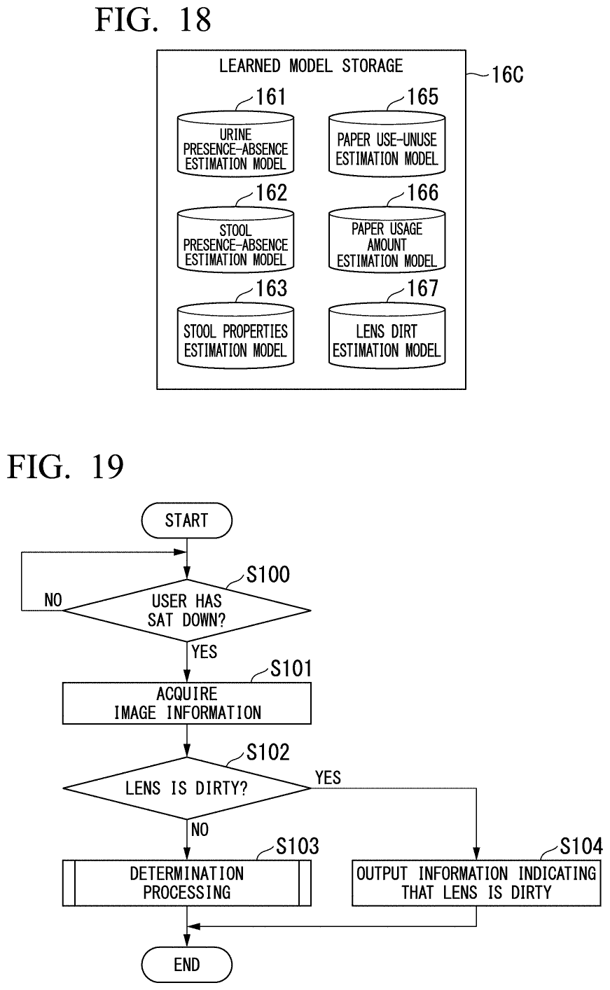

The determination device 10C includes a learned model storage 16D. As illustrated in FIG. 18, the learned model storage 16C includes a lens dirt estimation model 167. The lens dirt estimation model 167 is a learned model that has learned a correspondence relationship between an image and presence-absence of lens dirt in an image pickup device that has photographed the image, and is created by performing learning using learning data that associates a subject image with information indicating presence-absence of lens dirt determined from the image. The lens dirt is, for example, binary information indicating whether or not the lens is dirty, or information indicating a plurality of levels that depend on the degree of lens dirt. As the method of determining presence-absence of lens dirt, for example, a person in charge of creating learning data may determine presence-absence of lens dirt in an image.

The analyzer 12 estimates presence-absence of lens dirt in an image pickup device that has photographed an image by using the lens dirt estimation model 167. The analyzer 12 sets an output obtained by inputting a subject image into the lens dirt estimation model 167 as an estimation result of estimating presence-absence of lens dirt in the subject image.

The determiner 13 determines presence-absence of lens dirt in the subject image by using an analysis result obtained from the analyzer 12. For example, when the determiner 13 has determined that there is lens dirt in the subject image through use of the analyzer 12, the determiner 13 determines that there is lens dirt in the subject image. When the determiner 13 has determined that there is no lens dirt in the subject image through use of the analyzer 12, the determiner 13 determines that there is no lens dirt in the subject image.

When the determiner 13 estimated that there is lens dirt in the subject image through use of the analyzer 12, the determiner 13 may output information indicating that there is lens dirt via the outputter 14.

When the determiner 13 has determined that there is no lens dirt in the subject image through use of the analyzer 12, the determiner 13 may determine, for example, presence-absence of urine, presence-absence of stools, the properties of stools, the amount of usage of paper, and the flushing method (hereinafter referred to as “presence-absence of urine and the like”). As a result, it is possible to perform determination by using an estimation result such as presence-absence of urine and the like, which are estimated from an image that does not include lens dirt. Therefore, it is possible to use a more accurate estimation result compared with the case of using a result estimated from an image including lens dirt.

Now, a flow of processing to be executed by the determination device 10C is described with reference to FIG. 19. In Step S100, the determination device 10C determines whether or not the user of the toilet device 3 has sat on the toilet 30 through communication with the toilet device 3. When the determination device 10C has determined that the user has sat on the toilet 30, in Step S101, the determination device 10C acquires image information.

Next, in Step S102, the determination device 10C determines whether or not there is lens dirt. The determination device 10C determines presence-absence of lens dirt based on an output obtained by inputting an image into the lens dirt estimation model 167. When there is no lens dirt, in Step S103, the determination device 10C performs determination processing. The determination processing is similar to the processing illustrated in Step S12 of FIG. 4, and thus description thereof is omitted here. When there is lens dirt, in Step S104, the determination device 10C outputs information indicating that there is lens dirt.

In the above description, in Step S103, determination processing is performed only when there is no lens dirt as an example. However, this disclosure is not limited thereto. Even when there is lens dirt, the determination device 10C may perform determination processing in consideration of that dirt. In this case, the determination device 10C sets a learned model that performs determination as a model adapted for the case in which there is lens dirt. Specifically, the determination device 10C performs determination processing by using a learned model that has learned a correspondence relationship between an image for learning, which includes dirt due to an image pickup device or an image pickup environment, and a determination result of the determination matter relating to excretion, the learned model learned by machine learning using a neural network.

For example, the urine presence-absence estimation model 161 is a learned model that has learned a correspondence relationship between an image in which lens dirt is photographed and presence-absence of urine. That is, the urine presence-absence estimation model 161 is a model that is created by performing learning using learning data that associates an image in which lens dirt is photographed together with the situation of the toilet bowl 32 after excretion with information indicating presence-absence of urine determined from the image. For example, the stool presence-absence estimation model 162 is a learned model that has learned a correspondence relationship between an image in which lens dirt is photographed with information indicating presence-absence of stools. That is, the stool presence-absence estimation model 162 is a model that is created by performing learning using learning data that associates an image in which lens dirt is photographed together with the situation of the toilet bowl 32 after excretion with information indicating presence-absence of stools determined from the image. The stool properties estimation model 163, the paper use-unuse estimation model 165, and the paper usage amount estimation model 166 perform processing in a similar way.

In Step S104 described above, information indicating that there is lens dirt is output when there is lens dirt, but such information may be output to any functional unit as an output destination.

For example, the determination device 10 may output information indicating that there is lens dirt in a remote controller that is operated at the time of washing the human's bottom, for example. In this case, for example, the remote controller lights up a lens dirt mark among various kinds of marks included in the remote controller. Various kinds of marks included in the remote controller are marks that notify of a result of sensing the state of the toilet device 3, and are, for example, marks for notifying of the temperature setting of the toilet seat, the strength of washing the human's bottom, whether or not the power source of the remote controller is turned on, running out of the battery of the remote controller, presence-absence of lens dirt, and the like.

The determination device 10 may notify the user of information indicating that there is lens dirt by sound, display, or the like. In this case, the determination device 10C or the remote controller includes a speaker that outputs sound or a display for displaying an image. As a result, it is possible to cause the user to recognize the fact that there is lens dirt, prompt the user to clean the image pickup device 4 and the surroundings of the image pickup device 4, and maintain a clean state of the image pickup device 4 in which there is no lens dirt.

When the toilet device 3 has a cleaning function of cleaning the image pickup device 4 provided in the toilet device 3 and the surroundings of the image pickup device 4, the toilet device 3 may notify a controller that controls the lens cleaning function of the fact that there is lens dirt. The controller that controls the lens cleaning function may be provided in the toilet 30, or may be provided in a remote controller (not shown) or the like of the toilet 30, which is separate from the toilet 30. When the controller receives a notification indicating that there is lens dirt from the determination device 10C, the controller operates the lens cleaning function to clean the lens. As a result, it is possible to remove lens dirt, and photograph an image that does not include lens dirt.

As described above, in the determination device 10C according to the fourth embodiment, the determination matter includes presence-absence of lens dirt in the image pickup device that has photographed a subject image. As a result, the determination device 10C according to the fourth embodiment is capable of determining presence-absence of lens dirt. Therefore, it is possible to perform processing that depends on presence-absence of lens dirt.

In the determination device 10C according to some embodiments, the determination matter includes at least any one of presence-absence of urine, presence-absence of stools, and the properties of stools. When it is estimated that there is lens dirt through use of the analyzer 12, the determiner 13 does not perform determination of any one of presence-absence of urine, presence-absence of stools, and the properties of stools. As a result, the determination device 10C according to some embodiments is capable of preventing determination of the properties of stools or the like when there is lens dirt. Therefore, it is possible to perform determination more accurately compared with the case of performing determination also when there is lens dirt.

The timing of determining presence-absence of lens dirt is not limited to the timing of performing determination of a determination matter. Even when it is not detected that the user has sat on the toilet seat of the toilet device 3, the internal space 34 of the toilet bowl 32 may be photographed and presence-absence of lens dirt may be determined based on the photographed image at any timing. For example, presence-absence of lens dirt may be determined periodically, for example, once a day.

In the determination device 10C according to some embodiments, the determination matter includes at least any one of presence-absence of urine, presence-absence of stools, and the properties of stools. When it is estimated that there is lens dirt through use of the analyzer 12, the determiner 13 may determine any one of presence-absence of urine, presence-absence of stools, and the properties of stools by using a model that considers lens dirt. The model that considers lens dirt is a learned model that has learned a correspondence relationship between an image for learning, which includes dirt due to an image pickup device or an image pickup environment, and a determination result of the determination matter relating to excretion, the learned model learned by machine learning using a neural network. As a result, even when there is lens dirt, the determination device 10C is capable of determining the properties of stools or the like in consideration of the fact that lens dirt is photographed. Therefore, when there is lens dirt, it is possible to perform determination more accurately compared with the case of performing determination without considering the fact that there is lens dirt.

In the determination device 10C, when it is estimated that there is lens dirt through use of the analyzer 12, the determiner 13 may output information indicating that there is lens dirt via the outputter 14. As a result, for example, it is possible to output the information indicating that there is lens dirt to a remote controller, and light up a lens dirt mark of the remote controller. Alternatively, it is possible to output information indicating that there is lens dirt by sound, or display the information indicating that there is lens dirt on an image. In this manner, it is possible to cause the user to recognize the fact that there is lens dirt, prompt the user to clean the lens, and maintain a clean state of the lens in which there is no lens dirt. Alternatively, it is possible to maintain a clean state of the lens in which there is no lens dirt by outputting information indicating that there is lens dirt to a controller that controls a lens cleaning function of the toilet device 3 and operating the lens cleaning function. In the description given above, the device serving as an output destination to which information indicating that there is lens dirt is output is a remote controller. However, this disclosure is not limited thereto. The output destination may include any device that may cope with lens dirt. For example, the output destination may be a user terminal of a user who uses a toilet, a cleaning company terminal of a cleaning company that cleans the toilet, or a facility manager terminal of a facility manager who manages a facility in which the toilet is provided.

In the description given above, the details of notification by the determination device 10C are the fact that there is lens dirt as an example. However, this disclosure is not limited thereto. The determination device 10C may notify of any details that depend on the output destination based on the result of determination by the determiner 13. For example, the determination device 10C may notify the user of the fact that the toilet is dirty, the degree of dirt, and the necessity of cleaning the toilet. The determination device 10C may notify the user sequentially of the progress after giving the notification that the lens is dirty. For example, when the determination device 10C notifies a plurality of recipients that the lens is dirty, and one recipient replies that the toilet has been cleaned, the determination device 10C may notify the plurality of previously notified recipients that the cleaning has been completed.

All or a part of the processing performed by the determination devices 10, 10A, 10B, and 10C in the above-mentioned embodiments may be implemented by a computer. In that case, all or a part of the processing may be implemented by recording a program for implementing this function into a computer-readable recording medium, causing a computer system to read the program recorded in this recording medium, and executing the program. The phrase “computer system” includes hardware such as an operating system and peripheral devices. The phrase “computer-readable storage medium” refers to a portable medium such as a flexible disk, an optical disk, a ROM, or a CD-ROM, or a storage device such as a hard disk built into a computer system. Furthermore, the phrase “computer-readable storage medium” may include a device that holds a program for a certain period of time, such as a communication line that dynamically holds a program for a short period of time in a case where the program is transmitted over a network such as the Internet or a communication line such as a telephone line, or a volatile memory in a computer system that serves as a server or a client in that case. The above-mentioned program may be used to implement a part of the above-mentioned functions, or may be used to implement the above-mentioned functions in combination with a program ready recorded in the computer system, or may be used to implement the above-mentioned functions by using a programmable logic device such as an FPGA.

1. A determination device, comprising:
an image information acquirer configured to acquire image information of a subject image obtained by photographing an internal space of a toilet bowl in excretion;
an estimator configured to perform estimation regarding a determination matter relating to excretion by inputting the image information to a learned model, the learned model having learned a correspondence relationship between an image for learning and a determination result of the determination matter relating to excretion by machine learning using a neural network, the image for learning representing an internal space of a toilet bowl in excretion; and
a determiner configured to perform determination regarding the determination matter of the subject image based on an estimation result obtained by the estimator.

2. The determination device of claim 1, wherein the subject image is an image obtained by photographing the internal space of the toilet bowl after excretion.

3. The determination device of claim 1, wherein the determination matter includes at least one of presence-absence of urine, presence-absence of stools, and properties of stools.

4. The determination device of claim 1, wherein the determination matter includes use-unuse of paper in excretion and an amount of usage of paper in a case where paper has been used.

5. The determination device of claim 1, wherein the determiner determines a flushing method for flushing a toilet under a situation indicated by the subject image.

6. The determination device of claim 5,
wherein the determination matter includes at least one of properties of stools and an amount of usage of paper in excretion,
the estimator estimates at least any one of properties of stools in the subject image and the amount of usage of paper in excretion in the subject image, and
the determiner determines the flushing method for flushing the toilet under the situation indicated by the subject image based on at least any one of the properties of stools and the amount of usage of paper in excretion, the properties of stools and the amount of usage of paper in excretion are estimated by the estimator.

7. The determination device of claim 1, wherein the determination matter includes determination of whether or not excretion has been performed.

8. The determination device of claim 1, wherein
the determination device is configured to be connected with a toilet device including the toilet bowl, a toilet seat and a human's bottom washing device the determiner performs determination regarding the determination matter at predetermined time intervals until a predetermined end condition is satisfied after a predetermined start condition is satisfied,
the start condition is to detect that a user has sat on the toilet seat of the toilet device, and
the end condition is at least any one of use of the human's bottom washing device of the toilet device, an operation of flushing the toilet bowl of the toilet device, and detection of the user standing up from the toilet seat of the toilet device.

9. The determination device of claim 1, wherein the determination matter includes determination of whether or not dirt is photographed in the subject image, the dirt is due to an image pickup device or an image pickup environment.

10. The determination device of claim 9, wherein
the determination matter includes at least any one of presence-absence of urine, presence-absence of stools, and properties of stools, and
the determiner does not perform determination of any one of presence-absence of urine, presence-absence of stools, and properties of stools when the estimator has estimated that the dirt is photographed.

11. The determination device of claim 9, wherein
the determination matter includes at least any one of presence-absence of urine, presence-absence of stools, and properties of stools, and
the determiner performs any one of determination of presence-absence of urine, presence-absence of stools, and properties of stools by using a learned model when the estimator has estimated that the dirt is photographed, the learned model has learned a correspondence relationship between the image for learning and a determination result of the determination matter relating to excretion by machine learning using a neural network, the image for learning includes the dirt.

12. The determination device of claim 9, wherein
the determiner outputs information indicating dirt to a destination set in advance when the estimator has estimated that the dirt is photographed.

13. A determination method for determining a determination matter relating to excretion, the determination method comprising:
acquiring image information of a subject image obtained by photographing an internal space of a toilet bowl in excretion by an image information acquirer;
performing estimation regarding the determination matter of the subject image by an estimator by inputting the image information to a learned model, the learned model having learned a correspondence relationship between an image for learning and a determination result of the determination matter relating to excretion by machine learning using a neural network, the image for learning representing an internal space of a toilet bowl in excretion; and
performing, by a determiner, determination regarding the determination matter of the subject image based on an estimation result obtained by the estimator.

14. A non-transitory computer readable storage medium that stores a program for causing computer executable instructions, when executed by one or more computers, the one or more computers comprising:
acquiring image information of a subject image obtained by photographing an internal space of a toilet bowl in excretion;
performing estimation regarding the determination matter of the subject image by an estimator by inputting the image information to a learned model, the learned model having learned a correspondence relationship between an image for learning and a determination result of the determination matter relating to excretion by machine learning using a neural network, the image for learning representing an internal space of a toilet bowl in excretion; and
performing determination regarding the determination matter of the subject image based on a result of the estimation.
